(19) United States
(12) Patent Application Publication
(10) Pub. No.: US 2022/0064105 A1
(43) Pub. Date: Mar. 3, 2022

(54) CRYSTALLINE EDG-2 RECEPTOR ANTAGONIST AND METHODS OF MAKING

(71) Applicant: Sanofi, Paris (FR)

(72) Inventors: Josef PERNERSTORFER, Paris (FR); William ROCCO, Reading, PA (US); Jason BRITTAIN, El Cajon, CA (US)

(21) Appl. No.: 17/463,369

(22) Filed: Aug. 31, 2021

Related U.S. Application Data
(60) Provisional application No. 63/072,848, filed on Aug. 31, 2020.
(60) Provisional application No. 63/227,279, filed on Jul. 29, 2021.

(51) Int. Cl. C07C 235/54; A61K 9/20; A61K 9/08; A61K 9/00; A61K 9/48

(52) U.S. Cl. CPC C07C 235/54; A61K 9/2054; A61K 9/4825; A61K 9/0019; A61K 9/4866; A61K 9/08

(57) ABSTRACT

Described herein are crystalline forms of 2-(4-methoxy-3-(3-methylphenethoxy)benzamido)-2,3-dihydro-1H-indene-2-carboxylic acid and methods of making the same. Such forms of 2-(4-methoxy-3-(3-methylphenethoxy)benzamido)-2,3-dihydro-1H-indene-2-carboxylic acid are useful in the preparation of pharmaceutical compositions for the treatment of diseases or conditions that would benefit by administration with an EDG-2 receptor antagonist compound.

### CROSS-REFERENCE

This application claims benefit of U.S. Provisional Patent Application No. 63/072,848 filed on Aug. 31, 2020 and U.S. Provisional Patent Application No. 63/227,279 filed on Jul. 29, 2021; each of which is incorporated herein by reference in its entirety.

### FIELD OF THE INVENTION

Described herein are crystalline forms of an endothelial differentiation gene 2 (EDG-2) antagonist compound, as well as pharmaceutical compositions thereof, and methods of use thereof in the treatment of diseases or conditions that would benefit with treatment with an EDG-2 antagonist compound.

### BACKGROUND OF THE INVENTION

EDG-2 (also known as lysophosphatidic acid receptor 1, LPA₁ receptor, LPAR1) is a member of the G protein-coupled receptor family of integral membrane proteins that are important for lipid signaling. The LPA₁ receptor is a member of the G protein-coupled receptor family of integral membrane proteins that are important for lipid signaling. LPA₁ receptor antagonists are useful in the treatment of diseases or conditions for which abnormal LPA signaling plays a role, such as atherosclerosis, myocardial infarction, and heart failure.

### SUMMARY OF THE INVENTION

The present disclosure relates to various solid state forms of the LPA₁ receptor antagonist 2-(4-methoxy-3-(3-methylphenethoxy)benzamido)-2,3-dihydro-1H-indene-2-carboxylic acid and methods of making the same. Such forms of 2-(4-methoxy-3-(3-methylphenethoxy)benzamido)-2,3-dihydro-1H-indene-2-carboxylic acid are useful for modulating the activity of LPA₁ receptors in mammals that would benefit from such activity.

Described herein, in some embodiments, is Crystalline Form 1 of 2-(4-methoxy-3-(3-methylphenethoxy)benzamido)-2,3-dihydro-1H-indene-2-carboxylic acid (Compound I). In some embodiments, crystalline Form 1 of Compound I is characterized as having: an X-ray powder diffraction (XRPD) pattern substantially the same as shown in FIG. 1, as measured using Cu (Kα) radiation; or an X-ray powder diffraction (XRPD) pattern derived using Cu (Kα) radiation with peaks at 5.2±0.2° 2-Theta, 9.0±0.2° 2-Theta, 14.4±0.2° 2-Theta, and 17.7±0.2° 2-Theta, as measured using Cu (Kα) radiation; or a Fourier Transform IR Spectroscopy (FTIR) pattern with a peak at about 1739.6 cm⁻¹; or unit cell parameters substantially equal to the following at 293 K:

| Crystal System | triclinic |
|---|---|
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |

or a Solid State ¹³Carbon Nuclear Magnetic Resonance (ssNMR) spectrum substantially the same as shown in FIG. 4; or a Solid State ¹³Carbon Nuclear Magnetic Resonance (ssNMR) spectrum characterized by resonances (δc) at 23.35, 124.43, 126.78, 127.42, and 136.47 ppm; or combinations thereof.

Also described herein, in some embodiments, is Crystalline Form 2 of 2-(4-methoxy-3-(3-methylphenethoxy)benzamido)-2,3-dihydro-1H-indene-2-carboxylic acid (Compound I). In some embodiments, crystalline Form 2 of Compound I is characterized as having: an X-ray powder diffraction (XRPD) pattern substantially the same as shown in FIG. 6, as measured using Cu (Kα) radiation; or an X-ray powder diffraction (XRPD) pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation; or a Fourier Transform IR Spectroscopy (FTIR) pattern with a peak at about 1731.7 cm⁻¹; or unit cell parameters substantially equal to the following at 293 K:

| Crystal System | orthorhombic |
|---|---|
| Space Group | Pbca; Z = 8 |
| a (Å) | 6.2823(10) |
| b (Å) | 23.285(4) |
| c (Å) | 31.614(6) |
| α (°) | 90.00° |
| β (°) | 90.00° |
| γ (°) | 90.00° |
| V (Å³) | 4624.5(14) |
| Calculated Density (Mg/m³) | 1.280 |
| Unique Reflections | 4163 |

or a Solid State ¹³Carbon Nuclear Magnetic Resonance (ssNMR) spectrum substantially the same as shown in FIG. 8; or a Solid State ¹³Carbon Nuclear Magnetic Resonance (ssNMR) spectrum characterized by resonances (δc) at 20.59, 126.39, 128.34, and 137.69 ppm; or combinations thereof.

Also described herein, in some embodiments, is Crystalline Form 3 of 2-(4-methoxy-3-(3-methylphenethoxy)benzamido)-2,3-dihydro-1H-indene-2-carboxylic acid (Compound I). In some embodiments, crystalline Form 3 of Compound I is characterized as having: an X-ray powder diffraction (XRPD) pattern substantially the same as shown in FIG. 10, as measured using Cu (Kα) radiation; or an X-ray powder diffraction (XRPD) pattern with peaks at 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta, as measured using Cu (Kα) radiation; or a Fourier Transform IR Spectroscopy (FTIR) pattern with a peak at about 1722.0 cm⁻¹; or a Solid State ¹³Carbon Nuclear Magnetic Resonance (ssNMR) spectrum substantially the same as shown in FIG. 12; or a Solid State ¹³Carbon Nuclear Magnetic Resonance (ssNMR) spectrum characterized by resonances (δc) at 64.56, 67.67, 122.99, and 126.71 ppm; or combinations thereof.

Also described herein, in some embodiments, is Crystalline Form 4 of 2-(4-methoxy-3-(3-methylphenethoxy)benzamido)-2,3-dihydro-1H-indene-2-carboxylic acid (Compound I). In some embodiments, crystalline Form 4 of Compound I is characterized as having: an X-ray powder diffraction (XRPD) pattern substantially the same as shown in FIG. 13, as measured using Cu (Kα) radiation; or a Fourier Transform IR Spectroscopy (FTIR) pattern with a peak at about 1743.9 cm⁻¹; or combinations thereof.

Also described herein, in some embodiments, is the amorphous phase of 2-(4-methoxy-3-(3-methylphenethoxy)benzamido)-2,3-dihydro-1H-indene-2-carboxylic acid (Compound I) characterized as having: an XRPD pattern showing a lack of crystallinity, and/or a Solid State ¹³Carbon Nuclear Magnetic Resonance (ssNMR) spectrum substantially the same as shown in FIG. 16.

Also described herein, in some embodiments, is a pharmaceutical composition comprising a crystalline form Compound I and at least one pharmaceutically acceptable excipient. For example, in some embodiments, described herein is a pharmaceutical composition comprising Crystalline Form 1 and at least one pharmaceutically acceptable excipient. In some embodiments, the pharmaceutical composition is formulated for administration to a mammal by oral administration. In some embodiments, the pharmaceutical composition is formulated for administration to a mammal by oral administration in the form of a tablet, a pill, a capsule, a suspension, or a solution. In some embodiments, the pharmaceutical composition is in the form of a solid form pharmaceutical composition. In some embodiments, the pharmaceutical composition is in the form of a tablet, a pill, or a capsule. In some embodiments, the pharmaceutical composition is substantially free of Compound I impurities. In some embodiments, the pharmaceutical composition comprises less than about 1% w/w of Compound I impurities. In some embodiments, the Compound I impurities comprise one or more degradants of Compound I, one or more intermediates used in the synthesis of Compound I, or combinations thereof. In some embodiments, the Compound I impurities comprise one or more intermediates used in the synthesis of Compound I.

In some embodiments, described herein is a process for the preparation of the Compound I:

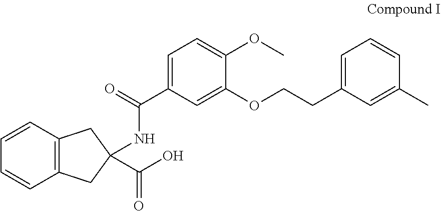 [SMILES: COc1ccc(C(=O)NC2(C(=O)O)Cc3ccccc3C2)cc1OCCc1cccc(C)c1]

comprising the steps of:
(1) contacting the compound of Formula 6:

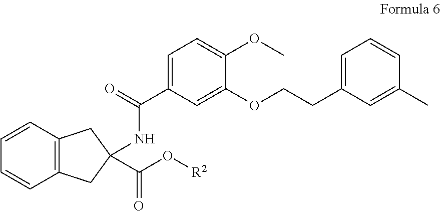

wherein R² is C₁-C₂₀ alkyl, C₁-C₂₀ alkenyl, C₃-C₁₀ cycloalkyl, or C₃-C₁₀ cycloalkenyl; with a hydroxide reagent having the formula M-OH in a suitable solvent to provide a compound of Formula 7:

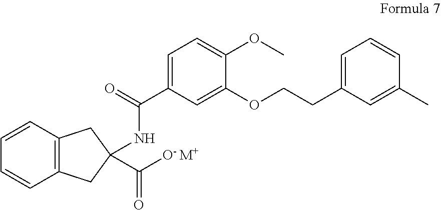

wherein M⁺ is Na⁺, K⁺, or Li⁺, and M-OH is NaOH, KOH, or LiOH, respectively; and (2) contacting the compound of Formula 7 with a suitable organic acid in a suitable solvent to provide Compound I.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 shows the X-ray powder diffraction (XRPD) pattern of Form 1.

FIG. 2 shows the Differential Scanning calorimetry (DSC) thermogram of Form 1.

FIG. 3 shows the Thermogravimetric Analysis (TGA) pattern of Form 1.

FIG. 4 shows the Solid State ¹³Carbon NMR Spectrum of Form 1.

FIG. 5 shows the Molecular Structure of Form 1.

FIG. 6 shows the X-ray powder diffraction (XRPD) pattern of Form 2.

FIG. 7 shows the Differential Scanning calorimetry (DSC) thermogram of Form 2.

FIG. 8 shows the Solid State ¹³Carbon NMR Spectrum of Form 2.

FIG. 9 shows the Molecular Structure of Form 2.

FIG. 10 shows the X-ray powder diffraction (XRPD) pattern of Form 3.

FIG. 11 shows the Differential Scanning calorimetry (DSC) thermogram of Form 3.

FIG. 12 shows the Solid State ¹³Carbon NMR Spectrum of Form 3.

FIG. 13 shows the X-ray powder diffraction (XRPD) pattern of Form 4.

FIG. 14 shows the Differential Scanning calorimetry (DSC) thermogram of Form 4.

FIG. 15 shows the Fourier Transform IR Spectroscopy (FTIR) pattern overlay of Forms 1, 2, 3, and 4.

FIG. 16 shows the Solid State ¹³Carbon NMR Spectrum of Amorphous Form.

FIG. 17 shows the XRPD pattern of Form 1 obtained with the Malvern Panalytical Empyrean diffractometer.

FIG. 18 shows the XRPD pattern of Form 2 obtained with the Malvern Panalytical Empyrean diffractometer.

FIG. 19 shows the XRPD pattern of Form 1 obtained with the Stoe Stadi P, G.52.SYS.S072 diffractometer.

FIG. 20 shows the XRPD pattern of Form 2 obtained with the Stoe Stadi P, G.52.SYS.S072 diffractometer.

FIG. 21 shows an overlay of the XRPD patterns of Form 1 (top XRPD) Form 2 (bottom XRPD) obtained with the Stoe Stadi P, G.52.SYS.S072 diffractometer.

FIG. 22 shows the XRPD pattern of Form 1 obtained with the PANalytical X'Pert PRO MPD diffractometer.

FIG. 23 shows the XRPD pattern of Form 2 obtained with the PANalytical X'Pert PRO MPD diffractometer.

FIG. 24 shows a comparison of XRPD patterns of forms 1 (top XRPD) and 2 (bottom XRPD), highlighting the Form 2 peaks used for the quantification of Form 2 in Form 1.

FIG. 25 shows XRPD overlays of the calibration standards used in the development of an XRPD limit test for determining Form 2 in Form 1 drug substance.

FIG. 26 shows the calibration curve used in the development of an XRPD limit test for determining Form 2 in Form 1 drug substance.

FIG. 27 shows the Raman spectrum for Form 1.

FIG. 28 shows the Raman spectrum for Form 2.

### DETAILED DESCRIPTION OF THE INVENTION

2-(4-methoxy-3-(3-methylphenethoxy)benzamido)-2,3-dihydro-1H-indene-2-carboxylic acid (Compound I) is a potent and selective LPA₁ receptor antagonist. The LPA₁ receptor is activated by lysophosphatidic acid (LPA). LPA₁ receptor antagonists are useful in the treatment of diseases or conditions for which abnormal LPA signaling plays a role, such as atherosclerosis, myocardial infarction, and heart failure.

### Compound I

Compound I is a potent selective orally available LPA₁ receptor antagonist that is useful in the treatment of a variety of diseases or conditions as described herein, such as fibrotic disease or conditions. In vivo, Compound I reversed dermal thickening and significantly inhibited myofibroblast differentiation and reduced collagen content in a mouse model of skin fibrosis. Mechanistic investigations showed that the antifibrotic effects of LPA₁ blockade could be mediated partly via inhibition of the Wnt signaling pathway. In the clinical setting, Compound I was well tolerated in patients with diffuse cutaneous systemic sclerosis SSc (dcSSc), demonstrated target engagement, and improved outcome measures (Y. Allanore et al. Arthritis & Rheumatology, Vol. 70, No. 10, October 2018, pp 1634-1643).

The preparation and uses of Compound I have been previously described (see, WO 2009/135590, U.S. Pat. Nos. 8,362,073, 8,445,530, 8,802,720, 9,328,071, each of which is incorporated by reference in its entirety).

Compound I refers to 2-(4-methoxy-3-(3-methylphenethoxy)benzamido)-2,3-dihydro-1H-indene-2-carboxylic acid, which has the chemical structure shown below:

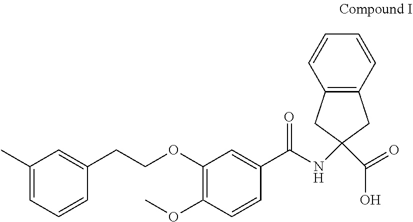 [SMILES: COc1ccc(C(=O)NC2(C(=O)O)Cc3ccccc3C2)cc1OCCc1cccc(C)c1]

In some embodiments provided herein, Compound I is crystalline.

In some embodiments provided herein, Compound I is a single crystalline form. In some embodiments provided herein, Compound I is a single crystalline form that is substantially free of any other crystalline form. In some embodiments, the crystalline solid form is a single solid state form, e.g. crystalline Form 1. In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05 w/w of any other crystalline form (e.g., Form 2) in a sample of crystalline Form 1. In some embodiments, “substantially free” means an undetectable amount (e.g., by XRPD analysis).

In some embodiments, crystallinity of a solid form is determined by X-Ray Powder Diffraction (XRPD). In some embodiments, crystallinity of a solid form is determined by solid state NMR. In some embodiments, crystallinity of a solid form is determined by Fourier Transform IR Spectroscopy (FTIR).

### Crystalline Form 1 of Compound I

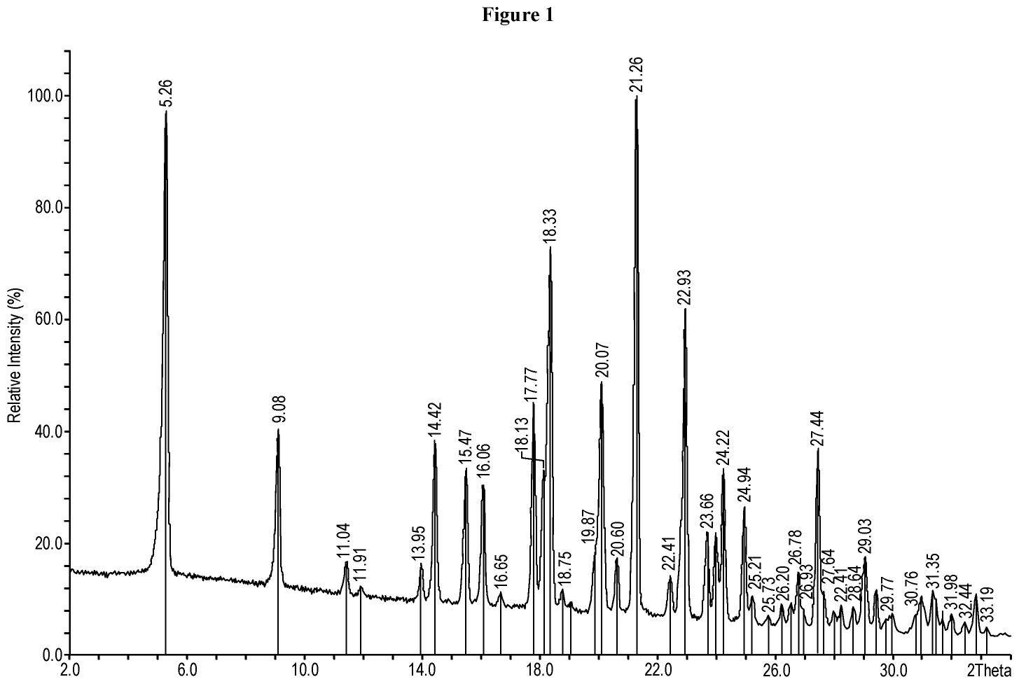

In one aspect, provided herein is crystalline Form 1 of 2-(4-methoxy-3-(3-methylphenethoxy)benzamido)-2,3-dihydro-1H-indene-2-carboxylic acid. Some embodiments provide a composition comprising crystalline Form 1 of 2-(4-methoxy-3-(3-methylphenethoxy)benzamido)-2,3-dihydro-1H-indene-2-carboxylic acid. In some embodiments, crystalline Form 1 of 2-(4-methoxy-3-(3-methylphenethoxy)benzamido)-2,3-dihydro-1H-indene-2-carboxylic acid is characterized as having:
an X-ray powder diffraction (XRPD) pattern substantially the same as shown in FIG. 1, as measured using Cu (Kα) radiation;
an X-ray powder diffraction (XRPD) pattern derived using Cu (Kα) radiation with peaks at 5.2±0.2° 2-Theta, 9.0±0.2° 2-Theta, 14.4±0.2° 2-Theta, and 17.7±0.2° 2-Theta, as measured using Cu (Kα) radiation;
a Fourier Transform IR Spectroscopy (FTIR) pattern with a peak at about 1739.6 cm⁻¹;
unit cell parameters substantially equal to the following at 293 K:

| Crystal System | triclinic |
|---|---|
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |

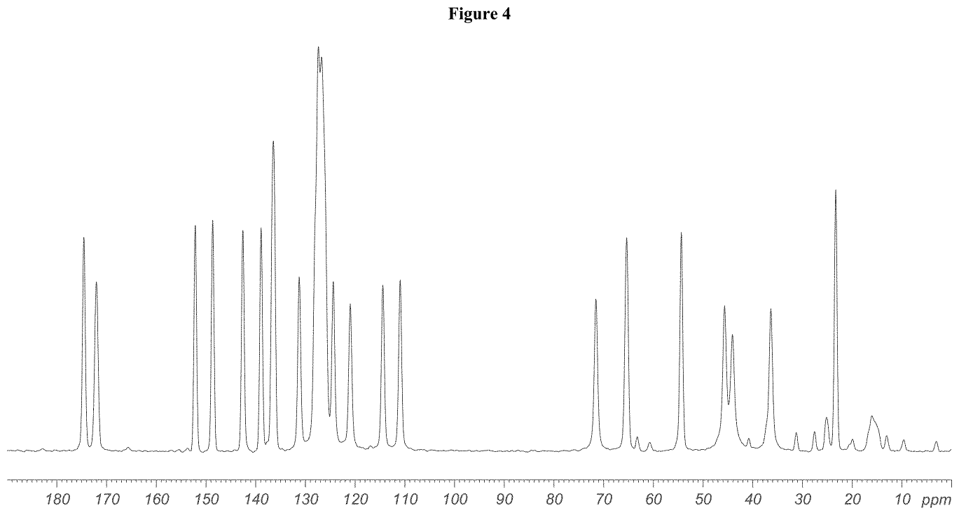

a Solid State ¹³Carbon Nuclear Magnetic Resonance (ssNMR) spectrum substantially the same as shown in FIG. 4;
a Solid State ¹³Carbon Nuclear Magnetic Resonance (ssNMR) spectrum characterized by resonances (δc) at 23.35, 124.43, 126.78, 127.42, and 136.47 ppm; or
combinations thereof.

In some embodiments, crystalline Form 1 of 2-(4-methoxy-3-(3-methylphenethoxy)benzamido)-2,3-dihydro-1H-indene-2-carboxylic acid (Compound I) has an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation.

In some embodiments, crystalline Form 1 of Compound I has an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation; and a Fourier Transform IR Spectroscopy (FTIR) pattern with a peak at about 1739.6 cm⁻¹.

In some embodiments, crystalline Form 1 of Compound I has an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation; and a Differential Scanning calorimetry (DSC) thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

In some embodiments, crystalline Form 1 of Compound I has an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation; and a Solid State ¹³Carbon Nuclear Magnetic Resonance (ssNMR) spectrum characterized by resonances (δc) at about 23.35 ppm, about 124.43 ppm, about 126.78 ppm, about 127.42 ppm, and about 136.47 ppm.

In some embodiments, crystalline Form 1 of Compound I has an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation; and a Solid State ¹³Carbon Nuclear Magnetic Resonance (ssNMR) spectrum characterized by resonances (δc) at about 23.35 ppm, about 124.43 ppm, about 126.78 ppm, about 127.42 ppm, and about 136.47 ppm; and a Differential Scanning calorimetry (DSC) thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

In some embodiments, crystalline Form 1 of Compound I has an X-ray powder diffraction (XRPD) pattern substantially the same as shown in FIG. 1, as measured using Cu (Kα) radiation.

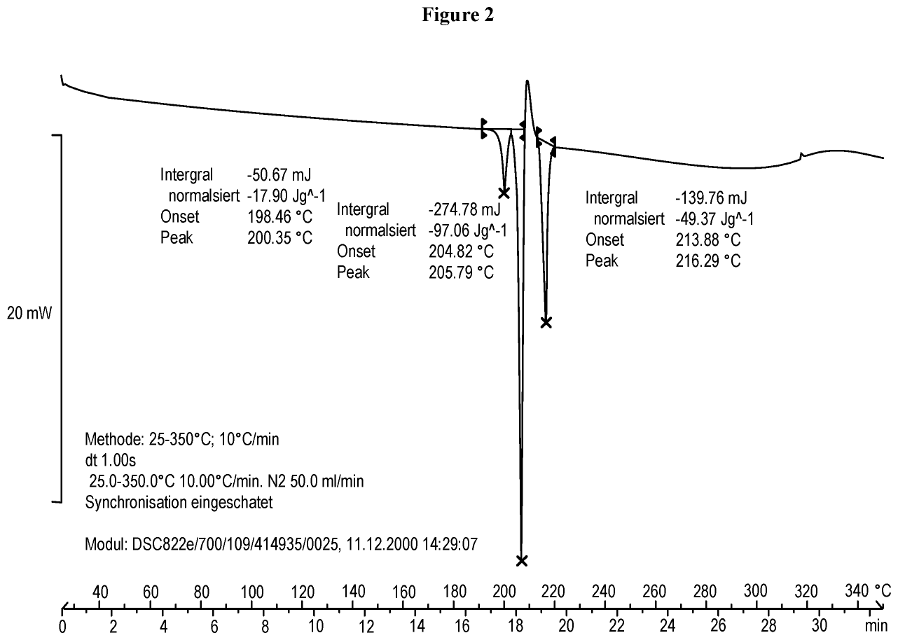

In some embodiments, crystalline Form 1 of Compound I has an X-ray powder diffraction (XRPD) pattern substantially the same as shown in FIG. 1, as measured using Cu (Kα) radiation; and a Differential Scanning calorimetry (DSC) thermogram substantially the same as shown in FIG. 2.

In some embodiments, crystalline Form 1 of Compound I has an X-ray powder diffraction (XRPD) pattern substantially the same as shown in FIG. 1, as measured using Cu (Kα) radiation; and a Fourier Transform IR Spectroscopy (FTIR) pattern with a peak at about 1739.6 cm⁻¹. In some embodiments, crystalline Form 1 of Compound I has an X-ray powder diffraction (XRPD) pattern substantially the same as shown in FIG. 1, as measured using Cu (Kα) radiation; and a Fourier Transform IR Spectroscopy (FTIR) pattern with a peak at about 1739.6 cm⁻¹; and a Differential Scanning calorimetry (DSC) thermogram substantially the same as shown in FIG. 2.

In some embodiments, crystalline Form 1 of Compound I has an X-ray powder diffraction (XRPD) pattern substantially the same as shown in FIG. 1, as measured using Cu (Kα) radiation; and a Solid State ¹³Carbon Nuclear Magnetic Resonance (ssNMR) spectrum substantially the same as shown in FIG. 4. In some embodiments, crystalline Form 1 of Compound I has an X-ray powder diffraction (XRPD) pattern substantially the same as shown in FIG. 1, as measured using Cu (Kα) radiation; and a Solid State ¹³Carbon Nuclear Magnetic Resonance (ssNMR) spectrum substantially the same as shown in FIG. 4; and a Differential Scanning calorimetry (DSC) thermogram substantially the same as shown in FIG. 2.

In some embodiments, crystalline Form 1 of 2-(4-methoxy-3-(3-methylphenethoxy)benzamido)-2,3-dihydro-1H-indene-2-carboxylic acid (Compound I) having unit cell parameters substantially equal to the following at 293 K:

| Crystal System | triclinic |
|---|---|
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |

In some embodiments, crystalline Form 1 of Compound I is characterized as having a Solid State ¹³Carbon Nuclear Magnetic Resonance (ssNMR) spectrum substantially the same as shown in FIG. 4. In some embodiments, crystalline Form 1 of Compound I is characterized as having a Solid State ¹³Carbon Nuclear Magnetic Resonance (ssNMR) spectrum substantially the same as shown in FIG. 4; and a Fourier Transform IR Spectroscopy (FTIR) pattern with a peak at about 1739.6 cm⁻¹. In some embodiments, crystalline Form 1 of Compound I is characterized as having a Solid State ¹³Carbon Nuclear Magnetic Resonance (ssNMR) spectrum substantially the same as shown in FIG. 4; and a Differential Scanning calorimetry (DSC) thermogram substantially the same as shown in FIG. 2.

In some embodiments, crystalline Form 1 of Compound I is characterized as having a Solid State ¹³Carbon Nuclear Magnetic Resonance (ssNMR) spectrum characterized by resonances (δc) at about 23.35 ppm, about 124.43 ppm, about 126.78 ppm, about 127.42 ppm, and about 136.47 ppm.

In some embodiments, crystalline Form 1 of Compound I is characterized as having a Solid State ¹³Carbon Nuclear Magnetic Resonance (ssNMR) spectrum characterized by resonances (δc) at about 23.35 ppm, about 124.43 ppm, about 126.78 ppm, about 127.42 ppm, and about 136.47 ppm; and a Differential Scanning calorimetry (DSC) thermogram substantially the same as shown in FIG. 2.

In some embodiments, crystalline Form 1 of Compound I is characterized as having a Solid State ¹³Carbon Nuclear Magnetic Resonance (ssNMR) spectrum characterized by resonances (δc) at about 23.35 ppm, about 124.43 ppm, about 126.78 ppm, about 127.42 ppm, and about 136.47 ppm; and a Fourier Transform IR Spectroscopy (FTIR) pattern with a peak at about 1739.6 cm⁻¹.

In some embodiments, crystalline Form 1 of Compound I is characterized as having a Fourier Transform IR Spectroscopy (FTIR) pattern with a peak at about 1739.6 cm⁻¹. In some embodiments, crystalline Form 1 of Compound I is characterized as having a Fourier Transform IR Spectroscopy (FTIR) pattern with a peak at about 1739.6 cm⁻¹; and a Differential Scanning calorimetry (DSC) thermogram substantially the same as shown in FIG. 2.

In some embodiments, crystalline Form 1 of Compound I is characterized as having a Fourier Transform IR Spectroscopy (FTIR) pattern with a peak at about 1739.6 cm⁻¹; and a Differential Scanning calorimetry (DSC) thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

In some embodiments, crystalline Form 1 of Compound I has a DSC thermogram substantially the same as shown in FIG. 2. In some embodiments, crystalline Form 1 has a DSC thermogram with one or more endothermic events having: an onset at about 198.5° C. and a peak at about 200.4° C.; an onset at about 204.8° C. and a peak at about 205.8° C.; and/or an onset at about 213.9° C. and a peak at about 216.3° C. In some embodiments, crystalline Form 1 has a DSC thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

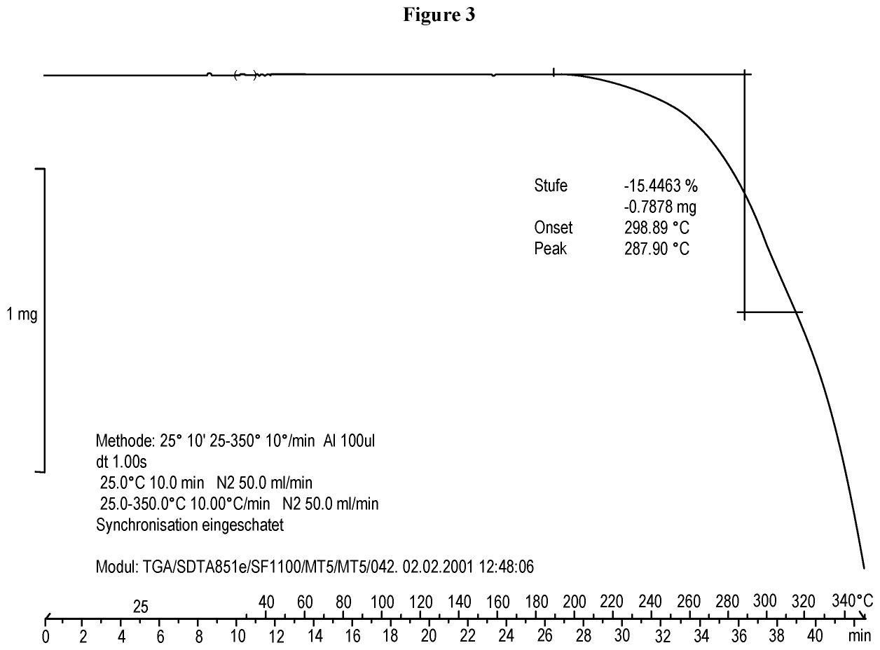

In some embodiments, crystalline Form 1 of 2-(4-methoxy-3-(3-methylphenethoxy)benzamido)-2,3-dihydro-1H-indene-2-carboxylic acid has a TGA pattern substantially the same as shown in FIG. 3. In some embodiments, crystalline Form 1 has a TGA pattern with a 15.4% w/w loss from about 287.9° C. to about 298.9° C. In some embodiments, crystalline Form 1 has a TGA pattern with less than 1% weight loss up to 200° C.

In some embodiments, crystalline Form 1 of Compound I has no reversible water uptake (˜−0.1% w/w) between 0 and 95% Relative Humidity (RH). In some embodiments, crystalline Form 1 of Compound I has no reversible water uptake between 0 and 95% Relative Humidity (RH). In some embodiments, crystalline Form 1 of Compound I has <1% w/w reversible water uptake between 0 and 95% Relative Humidity (RH). In some embodiments, crystalline Form 1 of Compound I has ˜−0.1% w/w reversible water uptake between 0 and 95% Relative Humidity (RH).

In some embodiments, crystalline Form 1 of Compound I has an FTIR spectrum with a peak at about 1739.6 cm⁻¹.

In some embodiments, crystalline Form 1 of 2-(4-methoxy-3-(3-methylphenethoxy)benzamido)-2,3-dihydro-1H-indene-2-carboxylic acid has an unchanged FTIR after storage at 75% RH and 80° C. over 7 days.

In some embodiments, crystalline Form 1 of Compound I has a crystal structure characterized by atomic coordinates substantially as in Table 2; wherein the measurement of the crystal structure is carried out at 293 K. In some embodiments, crystalline Form 1 has a crystal structure characterized by unit cell parameters substantially equal to: a=6.521(6) Å; b=10.548(9) Å; c=17.453(15) Å; α=104.080(16)°; β=92.430(16)°; γ=101.081(17)°; and having a triclinic space group=P1 (Z=2); wherein the measurement of the crystal structure is carried out at 293 K. In some embodiments, crystalline Form 1 has a crystal structure characterized by unit cell parameters substantially equal to: a=6.521(6) Å; b=10.548(9) Å; c=17.453(15) Å; α=104.080(16)°; β=92.430(16)°; γ=101.081(17)°; and having a triclinic space group=P1 (Z=2); wherein the measurement of the crystal structure is carried out at 293 K and is characterized by atomic coordinates substantially as in Table 2.

In some embodiments, crystalline Form 1 of Compound I has a ssNMR spectrum substantially the same as shown in FIG. 4. In some embodiments, crystalline Form 1 has a ssNMR spectrum characterized by resonances (δc) at 23.35, 124.43, 126.78, 127.42, and 136.47 ppm. In some embodiments, crystalline Form 1 has a ssNMR spectrum further characterized by resonances (δc) at 54.41, 65.40, 138.94, 142.61, 148.68, 152.19, and 174.59 ppm. In some embodiments, crystalline Form 1 has a ssNMR spectrum characterized by resonances (δc) at 23.35, 36.40, 44.12, 45.70, 54.41, 65.40, 71.58, 110.97, 114.45, 121.00, 124.43, 126.78, 127.42, 131.27, 136.47, 138.94, 142.61, 148.68, 152.19, 172.07, and 174.59 ppm.

In some embodiments, crystalline Form 1 of Compound I converts to crystalline Form 2 when slurried in solvent at a temperature of 60° C. or above. In some embodiments, crystalline Form 1 converts to crystalline Form 2 when slurried in MEK or 1-pentanol at a temperature of 60° C. or 70° C. In some embodiments, form conversion is determined by FTIR.

In some embodiments, crystalline Form 1 of Compound I is anhydrous.

### Crystalline Form 2 of Compound I

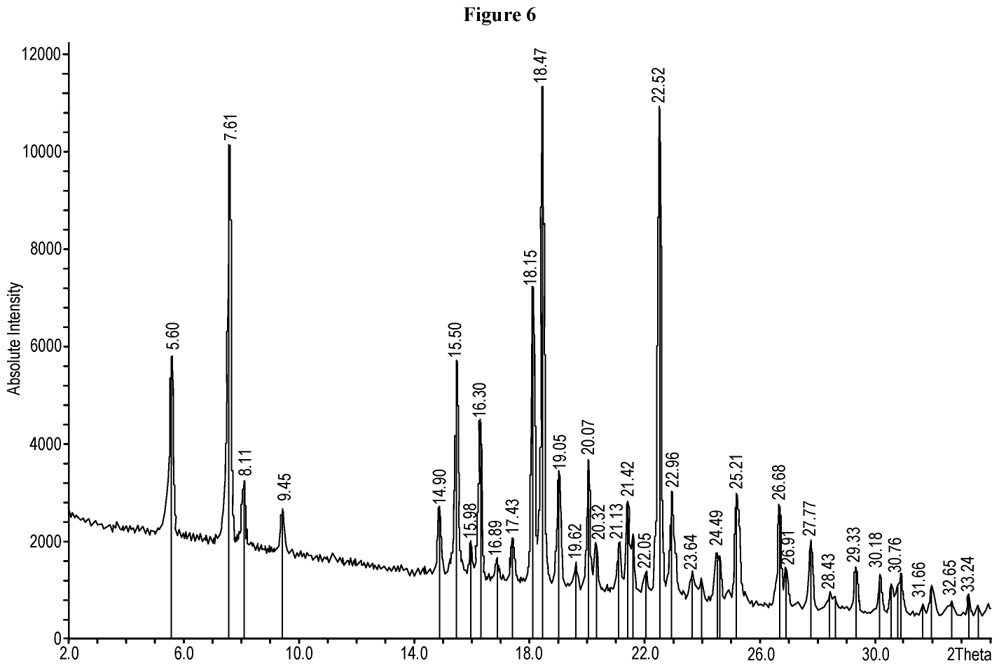

Also provided herein is crystalline Form 2 of 2-(4-methoxy-3-(3-methylphenethoxy)benzamido)-2,3-dihydro-1H-indene-2-carboxylic acid. Some embodiments provide a composition comprising crystalline Form 2 of 2-(4-methoxy-3-(3-methylphenethoxy)benzamido)-2,3-dihydro-1H-indene-2-carboxylic acid. In some embodiments, crystalline Form 2 of 2-(4-methoxy-3-(3-methylphenethoxy)benzamido)-2,3-dihydro-1H-indene-2-carboxylic acid is characterized as having:
an X-ray powder diffraction (XRPD) pattern substantially the same as shown in FIG. 6, as measured using Cu (Kα) radiation;
an X-ray powder diffraction (XRPD) pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation;
a Fourier Transform IR Spectroscopy (FTIR) pattern with a peak at about 1731.7 cm⁻¹;
unit cell parameters substantially equal to the following at 293 K:

| Crystal System | orthorhombic |
|---|---|
| Space Group | Pbca; Z = 8 |
| a (Å) | 6.2823(10) |
| b (Å) | 23.285(4) |
| c (Å) | 31.614(6) |
| α (°) | 90.00° |
| β (°) | 90.00° |
| γ (°) | 90.00° |
| V (Å³) | 4624.5(14) |
| Calculated Density (Mg/m³) | 1.280 |
| Unique Reflections | 4163 |

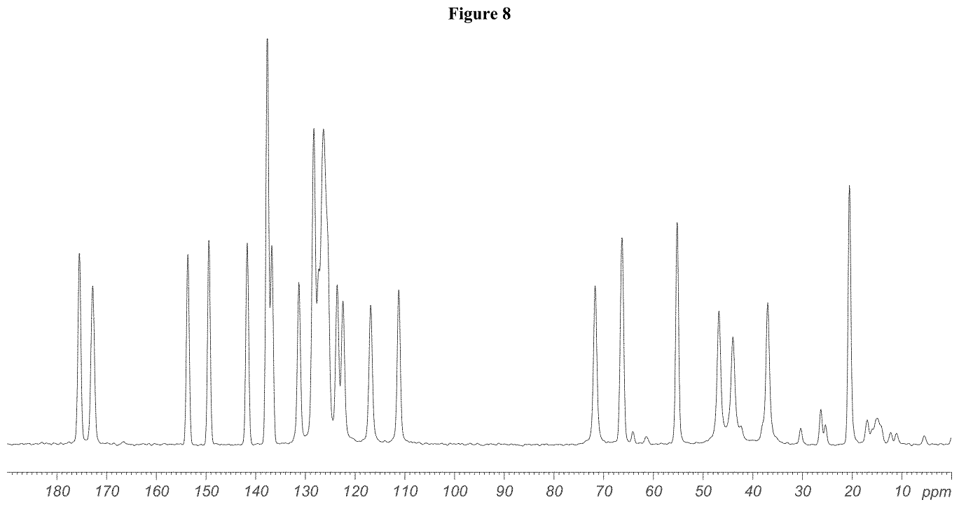

a Solid State ¹³Carbon Nuclear Magnetic Resonance (ssNMR) spectrum substantially the same as shown in FIG. 8;
a Solid State ¹³Carbon Nuclear Magnetic Resonance (ssNMR) spectrum characterized by resonances (δc) at 20.59, 126.39, 128.34, and 137.69 ppm; or
combinations thereof.

In some embodiments, crystalline Form 2 of Compound I is characterized as having an X-ray powder diffraction (XRPD) pattern substantially the same as shown in FIG. 6, as measured using Cu (Kα) radiation.

In some embodiments, crystalline Form 2 of Compound I is characterized as having an XRPD pattern substantially the same as shown in FIG. 6, as measured using Cu (Kα) radiation; and a Fourier Transform IR Spectroscopy (FTIR) pattern with a peak at about 1731.7 cm⁻¹.

In some embodiments, crystalline Form 2 of Compound I is characterized as having an XRPD pattern substantially the same as shown in FIG. 6, as measured using Cu (Kα) radiation; and a Solid State ¹³Carbon Nuclear Magnetic Resonance (ssNMR) spectrum substantially the same as shown in FIG. 8.

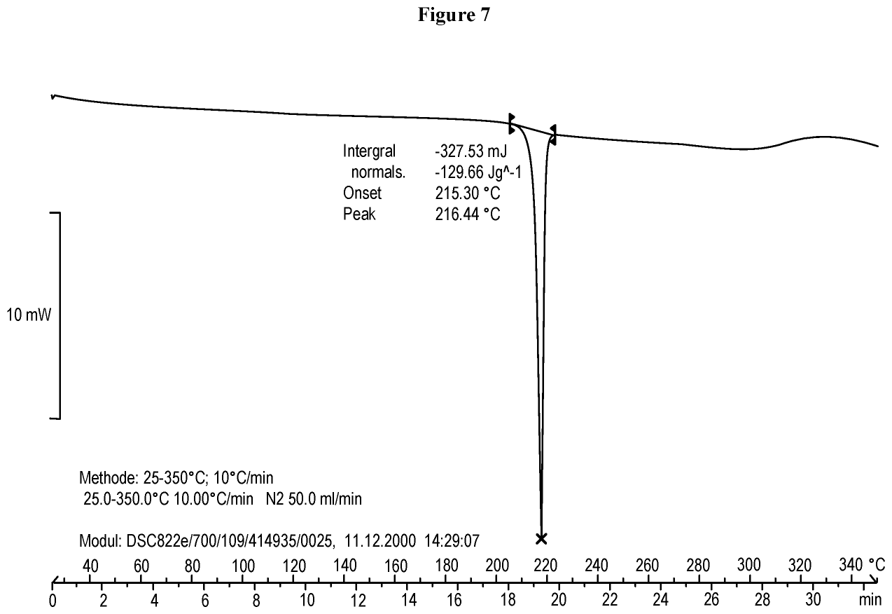

In some embodiments, crystalline Form 2 of Compound I is characterized as having an XRPD pattern substantially the same as shown in FIG. 6, as measured using Cu (Kα) radiation; and a Differential Scanning calorimetry (DSC) thermogram substantially the same as shown in FIG. 7.

In some embodiments, crystalline Form 2 of Compound I of 2-(4-methoxy-3-(3-methylphenethoxy)benzamido)-2,3-dihydro-1H-indene-2-carboxylic acid (Compound I) is characterized as having an X-ray powder diffraction (XRPD) pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation.

In some embodiments, crystalline Form 2 of Compound I is characterized as having an XRPD pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation; and a Differential Scanning calorimetry (DSC) thermogram with an endothermic event having an onset at about 215.3° C. and a peak at about 216.4° C.

In some embodiments, crystalline Form 2 of Compound I is characterized as having an XRPD pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation; and a Fourier Transform IR Spectroscopy (FTIR) pattern with a peak at about 1731.7 cm⁻¹.

In some embodiments, crystalline Form 2 of Compound I is characterized as having an XRPD pattern with peaks at 5.6±0.2° 2-Theta, 7.6±0.2° 2-Theta, 9.4±0.2° 2-Theta, 15.5±0.2° 2-Theta, and 16.3±0.2° 2-Theta, as measured using Cu (Kα) radiation; and a Solid State ¹³Carbon Nuclear Magnetic Resonance (ssNMR) spectrum characterized by resonances (δc) at 20.59, 126.39, 128.34, and 137.69 ppm.

In some embodiments, crystalline Form 2 of 2-(4-methoxy-3-(3-methylphenethoxy)benzamido)-2,3-dihydro-1H-indene-2-carboxylic acid (Compound I) is characterized as having unit cell parameters substantially equal to the following at 293 K:

| Crystal System | orthorhombic |
|---|---|
| Space Group | Pbca; Z = 8 |
| a (Å) | 6.2823(10) |
| b (Å) | 23.285(4) |
| c (Å) | 31.614(6) |
| α (°) | 90.00° |
| β (°) | 90.00° |
| γ (°) | 90.00° |
| V (Å³) | 4624.5(14) |
| Calculated Density (Mg/m³) | 1.280 |
| Unique Reflections | 4163 |

In some embodiments, crystalline Form 2 of Compound I is characterized as having a Solid State ¹³Carbon Nuclear Magnetic Resonance (ssNMR) spectrum substantially the same as shown in FIG. 8.

In some embodiments, crystalline Form 2 of Compound I is characterized as having a Solid State ¹³Carbon Nuclear Magnetic Resonance (ssNMR) spectrum substantially the same as shown in FIG. 8; and a Fourier Transform IR Spectroscopy (FTIR) pattern with a peak at about 1731.7 cm⁻¹.

In some embodiments, crystalline Form 2 of Compound I is characterized as having a Solid State ¹³Carbon Nuclear Magnetic Resonance (ssNMR) spectrum characterized by resonances (δc) at 20.59, 126.39, 128.34, and 137.69 ppm.

In some embodiments, crystalline Form 2 of Compound I is characterized as having a Solid State ¹³Carbon Nuclear Magnetic Resonance (ssNMR) spectrum characterized by resonances (δc) at 20.59, 126.39, 128.34, and 137.69 ppm; and a Differential Scanning calorimetry (DSC) thermogram with an endothermic event having an onset at about 215.3° C. and a peak at about 216.4° C.

In some embodiments, crystalline Form 2 of Compound I is characterized as having a Solid State ¹³Carbon Nuclear Magnetic Resonance (ssNMR) spectrum characterized by resonances (δc) at 20.59, 126.39, 128.34, and 137.69 ppm; and a Fourier Transform IR Spectroscopy (FTIR) pattern with a peak at about 1731.7 cm⁻¹.

In some embodiments, crystalline Form 2 of Compound I is characterized as having a Fourier Transform IR Spectroscopy (FTIR) pattern with a peak at about 1731.7 cm⁻¹.

In some embodiments, crystalline Form 2 has a TGA pattern with less than 1% weight loss up to 200° C.

In some embodiments, crystalline Form 2 of Compound I has a DSC thermogram substantially the same as shown in FIG. 7. In some embodiments, crystalline Form 2 has a DSC thermogram with an endothermic event having an onset at about 215.3° C. and a peak at about 216.4° C.

In some embodiments, crystalline Form 2 of Compound I has an FTIR spectrum with a peak at about 1731.7 cm⁻¹.

In some embodiments, crystalline Form 2 of Compound I has an unchanged FTIR after storage at 75% RH and 80° C. over 7 days.

In some embodiments, crystalline Form 2 of Compound I has a crystal structure characterized by atomic coordinates substantially as in Table 4; wherein the measurement of the crystal structure is carried out at 293 K. In some embodiments, crystalline Form 2 has a crystal structure characterized by unit cell parameters substantially equal to: a=6.2823(10) Å; b=23.285(4) Å; c=31.614(6) Å; α=90.00°; β=90.00°; γ=90.00°; and having an orthorhombic space group=Pbca (Z=8); wherein the measurement of the crystal structure is carried out at 293 K. In some embodiments, crystalline Form 2 has a crystal structure characterized by unit cell parameters substantially equal to: a=6.2823(10) Å; b=23.285(4) Å; c=31.614(6) Å; α=90.00°; β=90.00°; γ=90.00°; and having an orthorhombic space group=Pbca (Z=8); wherein the measurement of the crystal structure is carried out at 293 K and is characterized by atomic coordinates substantially as in Table 4.

In some embodiments, crystalline Form 2 of Compound I has a ssNMR spectrum substantially the same as shown in FIG. 8. In some embodiments, crystalline Form 2 has a ssNMR spectrum characterized by resonances (δc) at 20.59, 126.39, 128.34, and 137.69 ppm. In some embodiments, crystalline Form 2 has a ssNMR spectrum further characterized by resonances (δc) at 55.25, 66.34, 136.78, 141.73, 149.44, 153.68, and 175.49 ppm. In some embodiments, crystalline Form 2 has a ssNMR spectrum characterized by resonances (δc) at 20.59, 37.04, 44.03, 46.84, 55.25, 66.34, 71.74, 111.25, 116.90, 122.48, 123.63, 126.39, 128.34, 131.33, 136.78, 137.69, 141.73, 149.44, 153.68, 172.82, and 175.49 ppm.

In some embodiments, crystalline Form 2 of Compound I converts to crystalline Form 1 when slurried in solvent at a temperature of 50° C. or below. In some embodiments, crystalline Form 2 converts to crystalline Form 1 when slurried in MEK or methanol at a temperature of 40° C. or 50° C. In some embodiments, crystalline Form 2 converts to crystalline Form 1 when slurried in MEK at room temperature (˜25° C.). In some embodiments, form conversion is determined by FTIR.

### Crystalline Form 3 of Compound I

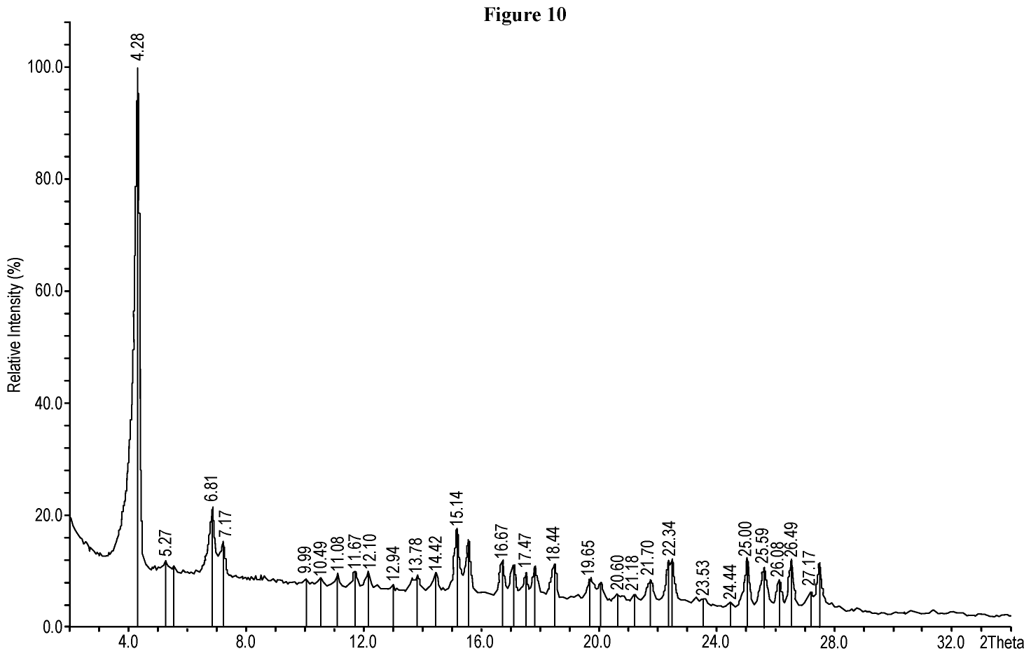

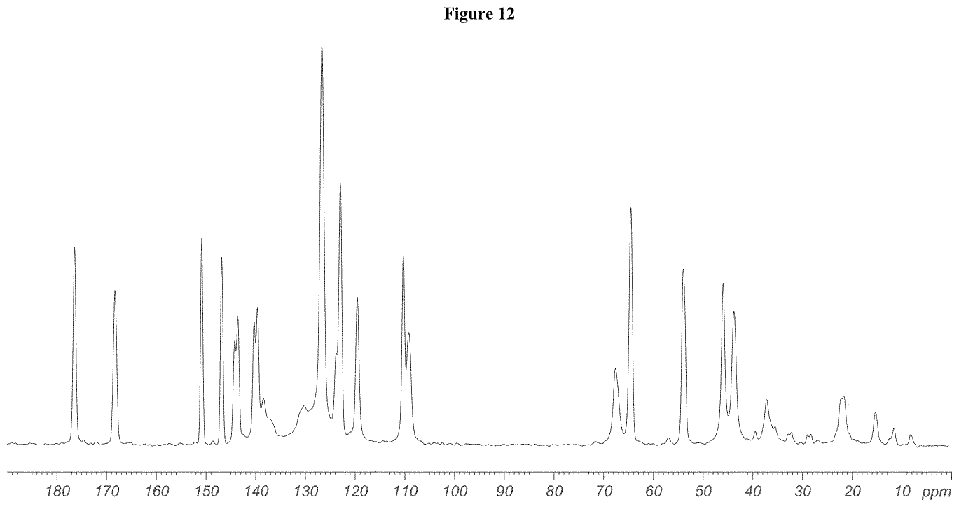

Also provided herein is the crystalline Form 3 of 2-(4-methoxy-3-(3-methylphenethoxy)benzamido)-2,3-dihydro-1H-indene-2-carboxylic acid. Some embodiments provide a composition comprising crystalline Form 3 of 2-(4-methoxy-3-(3-methylphenethoxy)benzamido)-2,3-dihydro-1H-indene-2-carboxylic acid. In some embodiments, crystalline Form 3 of 2-(4-methoxy-3-(3-methylphenethoxy)benzamido)-2,3-dihydro-1H-indene-2-carboxylic acid is characterized as having:
an X-ray powder diffraction (XRPD) pattern substantially the same as shown in FIG. 10, as measured using Cu (Kα) radiation;
an X-ray powder diffraction (XRPD) pattern with peaks at 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta, as measured using Cu (Kα) radiation;
a Fourier Transform IR Spectroscopy (FTIR) pattern with a peak at about 1722.0 cm⁻¹;
a Solid State ¹³Carbon Nuclear Magnetic Resonance (ssNMR) spectrum substantially the same as shown in FIG. 12;
a Solid State ¹³Carbon Nuclear Magnetic Resonance (ssNMR) spectrum characterized by resonances (δc) at 64.56, 67.67, 122.99, and 126.71 ppm; or
combinations thereof.

In some embodiments, crystalline Form 3 of Compound I is characterized as having an X-ray powder diffraction (XRPD) pattern substantially the same as shown in FIG. 10, as measured using Cu (Kα) radiation.

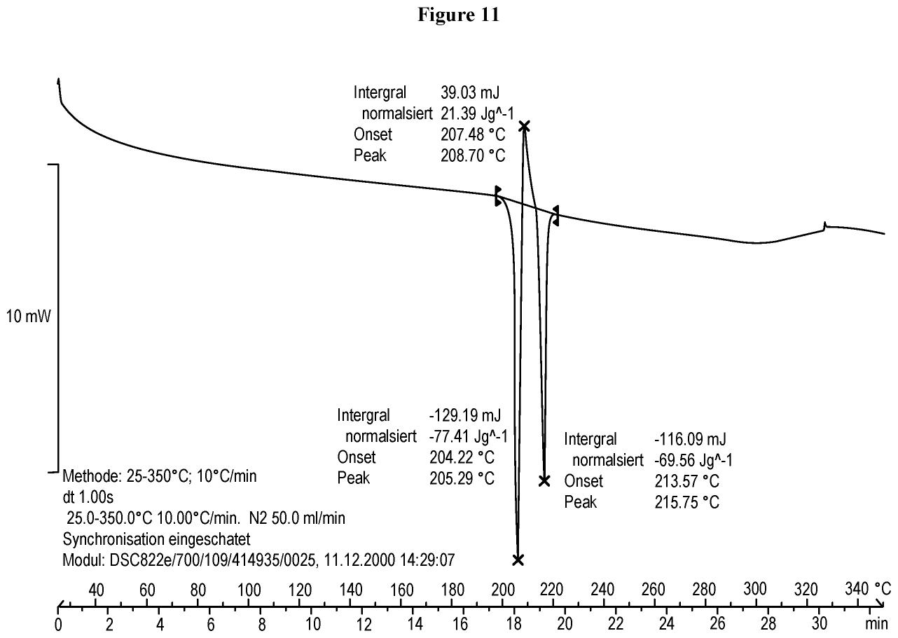

In some embodiments, crystalline Form 3 of Compound I is characterized as having an XRPD pattern substantially the same as shown in FIG. 10, as measured using Cu (Kα) radiation; and a Differential Scanning calorimetry (DSC) thermogram substantially the same as shown in FIG. 11.

In some embodiments, crystalline Form 3 of Compound I is characterized as having an XRPD pattern substantially the same as shown in FIG. 10, as measured using Cu (Kα) radiation; and a Fourier Transform IR Spectroscopy (FTIR) pattern with a peak at about 1722.0 cm⁻¹.

In some embodiments, crystalline Form 3 of Compound I of 2-(4-methoxy-3-(3-methylphenethoxy)benzamido)-2,3-dihydro-1H-indene-2-carboxylic acid (Compound I) is characterized as having an X-ray powder diffraction (XRPD) pattern with peaks at 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta, as measured using Cu (Kα) radiation.

In some embodiments, crystalline Form 3 of Compound I is characterized as having an XRPD pattern with peaks at 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta, as measured using Cu (Kα) radiation; and a Differential Scanning calorimetry (DSC) thermogram with one or more endothermic events having: an onset at about 204.2° C. and a peak at about 205.3° C.; and/or an onset at about 213.6° C. and a peak at about 215.8° C.

In some embodiments, crystalline Form 3 of Compound I is characterized as having an XRPD pattern with peaks at 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta, as measured using Cu (Kα) radiation; and a Fourier Transform IR Spectroscopy (FTIR) pattern with a peak at about 1722.0 cm⁻¹.

In some embodiments, crystalline Form 3 of Compound I is characterized as having a Fourier Transform IR Spectroscopy (FTIR) pattern with a peak at about 1722.0 cm⁻¹.

In some embodiments, crystalline Form 3 of Compound I is characterized as having a Solid State ¹³Carbon Nuclear Magnetic Resonance (ssNMR) spectrum substantially the same as shown in FIG. 12.

In some embodiments, crystalline Form 3 of Compound I is characterized as having a Solid State ¹³Carbon Nuclear Magnetic Resonance (ssNMR) spectrum characterized by resonances (δc) at 64.56, 67.67, 122.99, and 126.71 ppm.

In some embodiments, crystalline Form 3 has a TGA pattern with less than 1% weight loss up to 200° C.

In some embodiments, crystalline Form 3 of Compound I has a DSC thermogram substantially the same as shown in FIG. 11. In some embodiments, crystalline Form 3 has a DSC thermogram with one or more endothermic events having: an onset at about 204.2° C. and a peak at about 205.3° C.; and/or an onset at about 213.6° C. and a peak at about 215.8° C. In some embodiments, crystalline Form 3 has a DSC thermogram with two endothermic events having: an onset at about 204.2° C. and a peak at about 205.3° C.; and an onset at about 213.6° C. and a peak at about 215.8° C.

In some embodiments, crystalline Form 3 has an FTIR spectrum with a peak at about 1722.0 cm⁻¹. In some embodiments, crystalline Form 3 of 2-(4-methoxy-3-(3-methylphenethoxy)benzamido)-2,3-dihydro-1H-indene-2-carboxylic acid has an unchanged FTIR after storage at 75% RH and 80° C. over 7 days.

In some embodiments, crystalline Form 3 of Compound I has a ssNMR spectrum substantially the same as shown in FIG. 12. In some embodiments, crystalline Form 3 has a ssNMR spectrum characterized by resonances (δc) at 64.56, 67.67, 122.99, and 126.71 ppm. In some embodiments, crystalline Form 3 has a ssNMR spectrum further characterized by resonances (δc) at 110.33, 146.87, 150.90, and 176.47 ppm. In some embodiments, crystalline Form 3 has a ssNMR spectrum characterized by resonances (δc) at 43.81, 46.00, 54.01, 64.56, 67.67, 109.22, 110.33, 119.58, 122.99, 126.71, 139.68, 140.34, 143.63, 144.25, 146.87, 150.90, 168.32, and 176.47 ppm. In some embodiments, crystalline Form 3 has a ssNMR spectrum characterized by resonances (δc) at 21.72, 22.23, 43.81, 46.00, 54.01, 64.56, 67.67, 109.22, 110.33, 119.58, 122.99, 126.71, 130.28, 138.46, 139.68, 140.34, 143.63, 144.25, 146.87, 150.90, 168.32, and 176.47 ppm.

In some embodiments, crystalline Form 3 of Compound I converts to crystalline Form 1 when slurried in solvent at room temperature (˜25° C.). In some embodiments, crystalline Form 3 converts to crystalline Form 1 when slurried in methanol, MEK, methyl-THF, or ethyl acetate at room temperature (˜25° C.). In some embodiments, form conversion is determined by FTIR.

### Crystalline Form 4 of Compound I

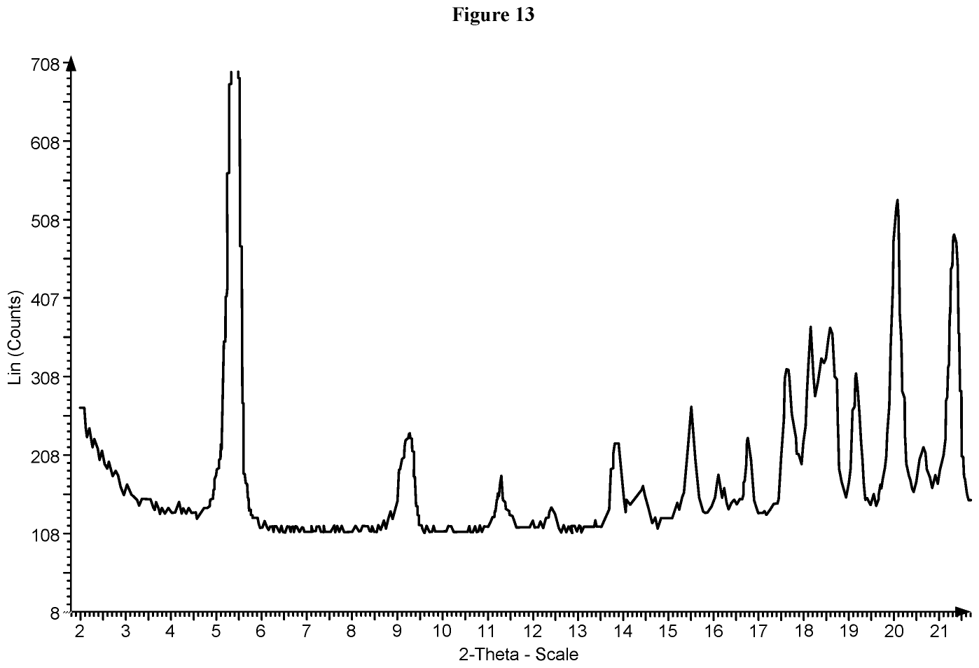

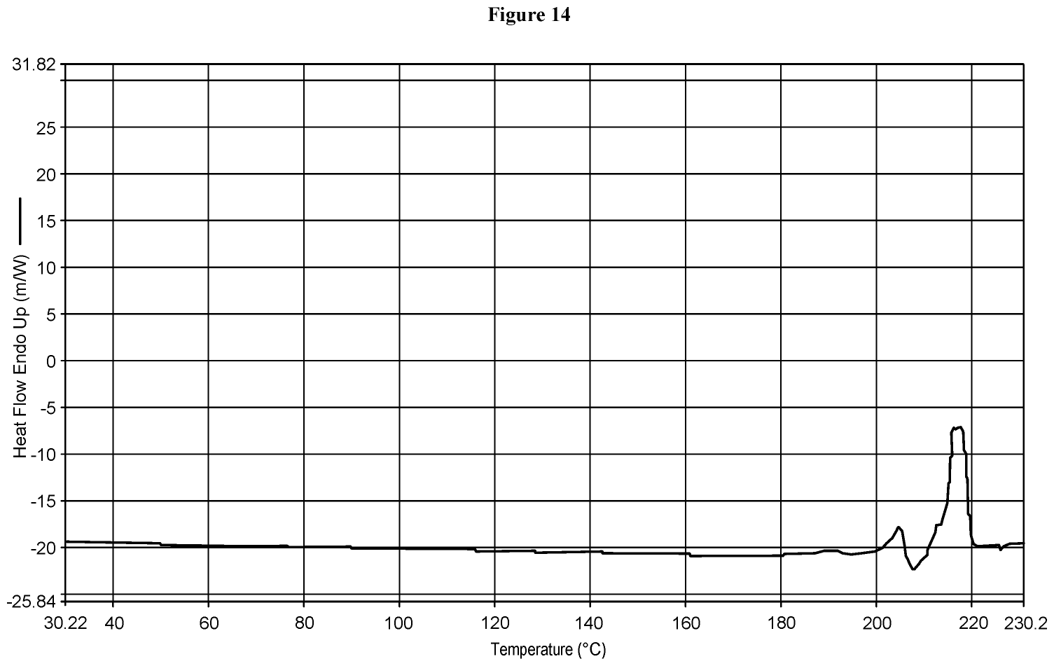

Also provided herein is the crystalline Form 4 of 2-(4-methoxy-3-(3-methylphenethoxy)benzamido)-2,3-dihydro-1H-indene-2-carboxylic acid. Some embodiments provide a composition comprising crystalline Form 4 of 2-(4-methoxy-3-(3-methylphenethoxy)benzamido)-2,3-dihydro-1H-indene-2-carboxylic acid. In some embodiments, crystalline Form 4 of 2-(4-methoxy-3-(3-methylphenethoxy)benzamido)-2,3-dihydro-1H-indene-2-carboxylic acid is characterized as having: an X-ray powder diffraction (XRPD) pattern substantially the same as shown in FIG. 13; a Differential Scanning calorimetry (DSC) thermogram substantially the same as shown in FIG. 14; a Fourier Transform IR Spectroscopy (FTIR) pattern with a peak at about 1743.9 cm⁻¹; or combinations thereof.

In some embodiments, crystalline Form 4 of 2-(4-methoxy-3-(3-methylphenethoxy)benzamido)-2,3-dihydro-1H-indene-2-carboxylic acid has an XRPD pattern substantially the same as shown in FIG. 13.

In some embodiments, crystalline Form 4 of 2-(4-methoxy-3-(3-methylphenethoxy)benzamido)-2,3-dihydro-1H-indene-2-carboxylic acid has a DSC thermogram substantially the same as shown in FIG. 14.

In some embodiments, crystalline Form 4 has an FTIR spectrum with a peak at about 1743.9 cm⁻¹.

In some embodiments, crystalline Form 4 has a TGA pattern with less than 1% weight loss up to 200° C.

### Amorphous Phase of Compound I

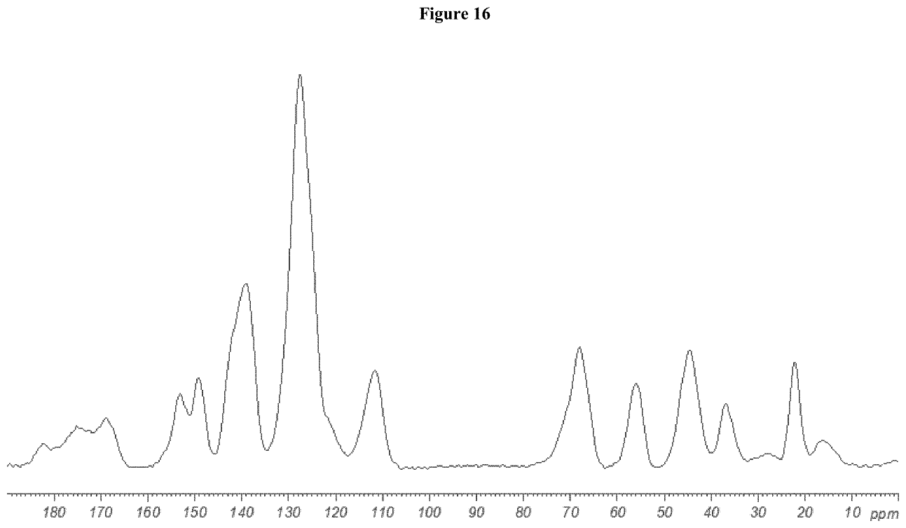

Also provided herein is the amorphous phase of 2-(4-methoxy-3-(3-methylphenethoxy)benzamido)-2,3-dihydro-1H-indene-2-carboxylic acid (Compound I). Some embodiments provide a composition comprising the amorphous phase of 2-(4-methoxy-3-(3-methylphenethoxy)benzamido)-2,3-dihydro-1H-indene-2-carboxylic acid (Compound I). In some embodiments, the amorphous phase of 2-(4-methoxy-3-(3-methylphenethoxy)benzamido)-2,3-dihydro-1H-indene-2-carboxylic acid (Compound I) is characterized as having an XRPD pattern showing a lack of crystallinity. In some embodiments, the amorphous phase of 2-(4-methoxy-3-(3-methylphenethoxy)benzamido)-2,3-dihydro-1H-indene-2-carboxylic acid (Compound I) is characterized as having a Solid State ¹³Carbon Nuclear Magnetic Resonance (ssNMR) spectrum substantially the same as shown in FIG. 16.

### Synthesis

Compounds described herein are synthesized using standard synthetic techniques or using methods known in the art in combination with methods described herein. Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In the reactions described, it may be necessary to protect reactive functional groups, for example hydroxy or amino groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

### Synthesis of Compound I

Disclosed herein are methods for the synthesis of Compound I, as outlined in Schemes 1-3.

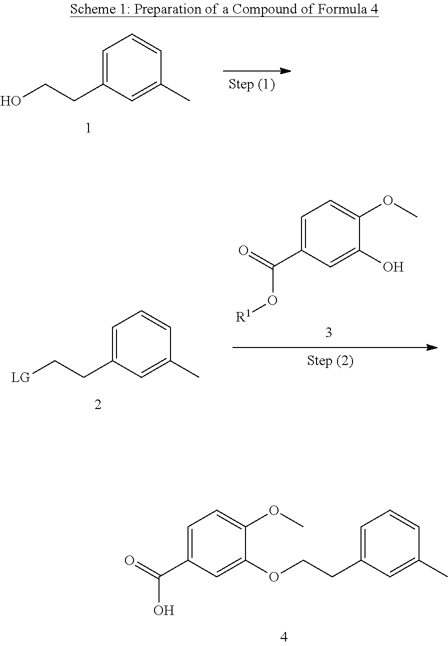

Briefly, in some embodiments, the primary alcohol of compound of Formula 1 is converted to a leaving group to yield the compound of Formula 2. In some embodiments, the compound of Formula 2 is reacted with the phenolic compound of Formula 3, followed by saponification to yield Compound 4.

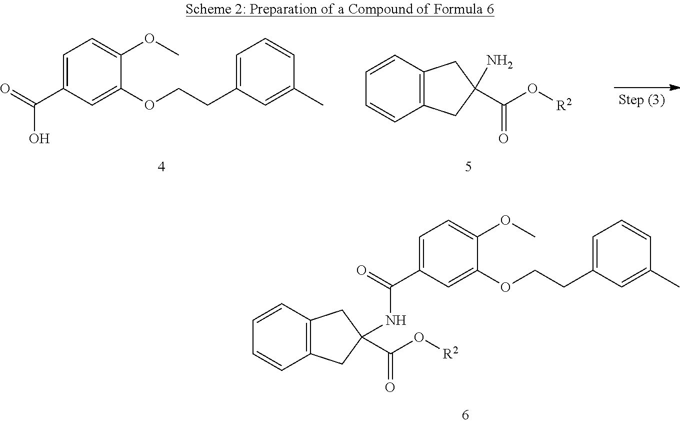

Briefly, in some embodiments, acid Compound 4 undergoes an amide bond formation reaction with the compound of Formula 5 to yield the compound of Formula 6.

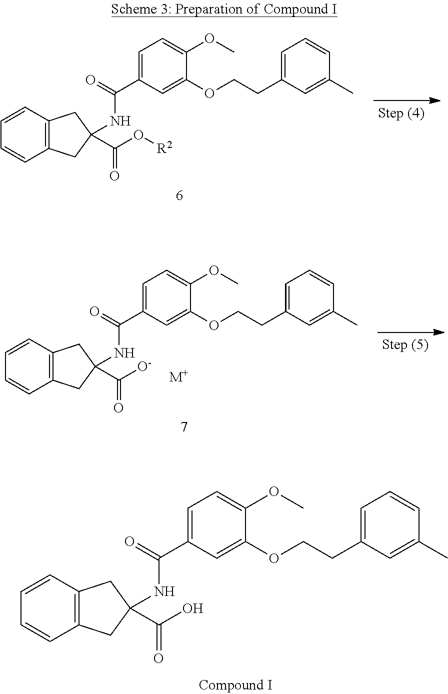

Briefly, the compound of Formula 6 undergoes a saponification reaction with NaOH, KOH, or LiOH to yield the salt of Formula 7. The salt of Formula 7 is acidified with a suitable organic acid to provide Compound I.

As disclosed herein, variables in Scheme 3 are defined as follows: LG is a suitable leaving group; R¹ is C₁-C₂₀ alkyl, C₁-C₂₀ alkenyl, C₃-C₁₀ cycloalkyl, or C₃-C₁₀ cycloalkenyl; R² is C₁-C₂₀ alkyl, C₁-C₂₀ alkenyl, C₃-C₁₀ cycloalkyl, or C₃-C₁₀ cycloalkenyl; and M⁺ is Na⁺, K⁺, or Li⁺.

In some embodiments, LG is a halogen, a sulfonate, or a sulfate. In some embodiments, LG is Cl, Br, I, mesylate, tosylate, or triflate. In some embodiments, LG is Cl, Br, I, —OTf, —OTs, or —OMs. In some embodiments, LG is a halogen. In some embodiments, LG is Cl, Br, or I. In some embodiments, LG is Br or I. In some embodiments, LG is a sulfonate. In some embodiments, LG is mesylate, tosylate, or triflate. In some embodiments, LG is —OTf, —OTs, or —OMs. In some embodiments, LG is —OMs.

In some embodiments, R¹ is C₁-C₁₀ alkyl, C₁-C₁₀ alkenyl, C₃-C₁₀ cycloalkyl, or C₃-C₁₀ cycloalkenyl. In some embodiments, le is C₁-C₂₀ alkyl or C₁-C₂₀ alkenyl. In some embodiments, le is C₁-C₁₀ alkyl or C₁-C₁₀ alkenyl. In some embodiments, le is C₁-C₆ alkyl or C₁-C₆ alkenyl. In some embodiments, le is C₁-C₆ alkyl. In some embodiments, le is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, le is methyl, ethyl, propyl, isopropyl, butyl, isobutyl, tert-butyl, isoamyl, pentyl, or hexyl. In some embodiments, le is methyl or ethyl. In some embodiments, le is methyl.

In some embodiments, R² is C₁-C₁₀ alkyl, C₁-C₁₀ alkenyl, C₃-C₁₀ cycloalkyl, or C₃-C₁₀ cycloalkenyl. In some embodiments, R² is C₁-C₂₀ alkyl or C₁-C₂₀ alkenyl. In some embodiments, R² is C₁-C₁₀ alkyl or C₁-C₁₀ alkenyl. In some embodiments, R² is C₁-C₆ alkyl or C₁-C₆ alkenyl. In some embodiments, R² is C₁-C₆ alkyl. In some embodiments, R² is methyl, ethyl, propyl, isopropyl, butyl, isobutyl, tert-butyl, isoamyl, pentyl, hexyl, heptyl, octyl, nonyl, terpenyl, bornyl, allyl, linalyl or geranyl. In some embodiments, R² is methyl, ethyl, propyl, isopropyl, butyl, isobutyl, tert-butyl, isoamyl, pentyl, or hexyl. In some embodiments, R² is methyl or ethyl. In some embodiments, R² is methyl.

In some embodiments, the compound of Formula 7 is not isolated between reaction steps 4 and 5. In some embodiments, steps 4 and 5 are performed in the same reaction vessel. In some embodiments, Compound I is crystallized from the reaction mixture to provide crystalline Form 1 of Compound I.

### Step 1: Synthesis of a Compound of Formula 2

In some embodiments, the alcohol —OH group of the compound of formula 1 is converted to a leaving group to yield the compound of Formula 2, by treatment with a suitable reagent in a suitable solvent.

In some embodiments, the suitable reagent is a halogenating agent, a sulfonating agent, or a sulfonyl chloride.

In some embodiments, the suitable reagent is a halogenating agent. In such embodiments, LG is a halogen. In some embodiments, LG is Cl, Br, or I. In some embodiments, LG is Br or I. In some embodiments, LG is Cl or Br. In some embodiments, LG is Br. In some embodiments, the suitable reagent is SOCl₂, PBr₃, or PCl₃, or the like. In some embodiments, the suitable reagent is PBr₃.

In some embodiments, the suitable reagent is a sulfonating agent. In such embodiments, LG is a sulfate.

In some embodiments, the suitable reagent is a sulfonyl chloride. In such embodiments, LG is a sulfonate. In some embodiments, the suitable reagent is tosyl chloride, mesyl chloride, or triflyl chloride, or the like. In such embodiments, LG is a tosylate, mesylate, or triflate, respectively, or the like. In some embodiments, the suitable reagent is mesyl chloride. In such embodiments, LG is mesylate.

In some embodiments, the suitable solvent is acetonitrile, dimethylformamide, diethyl ether, ethanol, tetrahydrofuran, isopropyl alcohol, 1,4-dioxane, toluene, water, or a combination thereof. In some embodiments, the suitable solvent is toluene.

In some embodiments, the reaction is performed at a low temperature. In some embodiments, the reaction is performed at a temperature below ambient temperature. In some embodiments, the reaction is performed at a temperature of about 0° C. to about 20° C. In some embodiments, the reaction is performed at about 5° C.

In some embodiments, Step 1 further comprises a suitable base. In some embodiments, the suitable base is pyridine, N-methylmorpholine, triethylamine, diisopropylethylamine, sec-butylamine, 1,2,2,6,6-pentamethylpiperidine, tributylamine, and 1,8-diazabicyclo[5.4.0]undec-7-ene (DBU), or the like. In some embodiments, the suitable base is triethylamine.

In some embodiments, the compound of Formula 2 is Compound 2a:

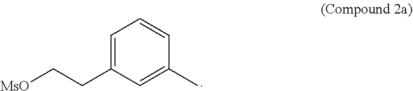

### Step 2: Synthesis of a Compound of Formula 4

In some embodiments, the compound of Formula 2 is reacted with a suitable base and the compound of Formula 3 in a suitable solvent (Step 2a), followed by saponification (Step 2b) to provide the compound of Formula 4.

In some embodiments, the suitable base for Step 2a is an amine base. In some embodiments, the suitable base is a tertiary amine base. In some embodiments, the suitable base is triethylamine, diisopropylethylamine, 1,2,2,6,6-pentamethylpiperidine, tributylamine, 1,8-diazabicycloundec-7-ene (DBU), or the like. In other embodiments, the suitable base is an inorganic base. In some embodiments, the suitable base is NaHCO₃, NaOAc, KOAc, Ba(OH)₂, Li₂CO₃, Na₂CO₃, K₂CO₃, Cs₂CO₃, Na₃PO₄, K₃PO₄, CsF, or the like. In some embodiments, the suitable base is K₂CO₃.

In some embodiments, the suitable solvent is acetonitrile, dimethylformamide, diethyl ether, ethanol, tetrahydrofuran, isopropyl alcohol, 1,4-dioxane, toluene, water, or a combination thereof. In some embodiments, the suitable solvent is ethanol.

In some embodiments, the reaction of Step 2a is performed at an elevated temperature. In some embodiments, the reaction is performed at the reflux temperature of the reaction mixture. In some embodiments, the reaction is performed at the boiling point of the solvent used. In some embodiments, the solvent is ethanol, and the reaction is performed at about 78-80° C. In some embodiments, the reaction is performed below the boiling point of the solvent used. In some embodiments, the reaction is performed at a temperature of about 60° C. to about 80° C. In some embodiments, the reaction is performed at about 65° C.

In some embodiments, Step 2a further comprises a phase transfer catalyst. In some embodiments, the phase transfer catalyst is tetrabutylammonium bromide, benzyltriethylammonium chloride, methyltricaprylammonium chloride, methyltributylammonium chloride, or methyltrioctylammonium chloride. In some embodiments, Step 2a further comprises tetrabutylammonium bromide.

In some embodiments, the saponification of Step 2b proceeds with a hydroxide reagent. In some embodiments, the hydroxide reagent is added directly to the reaction mixture of Step 2a.

In some embodiments, the hydroxide reagent is NaOH, KOH, or LiOH. In some embodiments, the hydroxide reagent is NaOH or KOH. In some embodiments, the hydroxide reagent is KOH. In some embodiments, the hydroxide reagent of Step 2b is provided as an aqueous solution. In some embodiments, the hydroxide reagent is about 0.1 M, about 0.5 M, about 1.0 M, about 2.0 M, about 5.0 M, about 10 M, or concentrated aqueous potassium hydroxide. In some embodiments, the hydroxide reagent is about 45% aqueous potassium hydroxide.

In some embodiments, the saponification of Step 2b is performed at an elevated temperature. In some embodiments, the saponification is performed at a temperature of about 60° C. to about 80° C. In some embodiments, the saponification is performed at about 65° C.

In some embodiments, the reaction mixture is acidified to provide Compound 4.

In some embodiments, the compound of Formula 3 is Compound 3a:

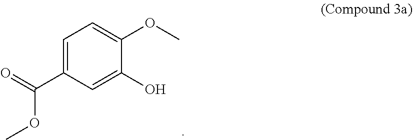

### Step 3: Synthesis of a Compound of Formula 6

In some embodiments, acid Compound 4 is reacted with the amine of the compound of Formula 5 to yield the amide compound of Formula 6 under amide bond forming conditions.

In some embodiments, the amide formation proceeds with a suitable reagent, a suitable base, and in a suitable solvent. In some embodiments, the suitable reagent is BOP, PyBOP, HATU, HBTU, pivaloyl chloride, or the like. In some embodiments, the suitable base is N-methylmorpholine, triethylamine, diisopropylethylamine, sec-butylamine, 1,2,2,6,6-pentamethylpiperidine, tributylamine, and 1,8-diazabicyclo[5.4.0]undec-7-ene (DBU), or the like. In some embodiments, the suitable solvent is acetonitrile, dimethylformamide, diethyl ether, ethanol, tetrahydrofuran, isopropyl alcohol, 1,4-dioxane, toluene, or a combination thereof.

In other embodiments, the acid of Compound 4 is converted to the acid chloride with a suitable reagent in a suitable solvent prior to reaction with the compound of Formula 5. In some embodiments, the suitable reagent is PCl₅, PCl₃, SOCl₂, oxalyl chloride (C₂O₂Cl₂), phosgene (COCl₂), triphosgene (C₃O₃Cl₆) or the like. In some embodiments, the suitable reagent is SOCl₂. In some embodiments, the reaction further comprises the use of N-methyl pyrrolidone (NMP), dimethylformamide (DMF), (chlormethylene)dimethylammonium chloride (Vilsmeier reagent) or analogues of the Vilsmeier reagent. In some embodiments, the reaction further comprises the use of N-methyl pyrrolidone (NMP). In some such embodiments, NMP is used in a catalytic amount, e.g., less than 0.2, less than 0.1, or less than 0.05 equivalents. In some embodiments, the reaction comprises about 0.05 equivalents of NMP. In some embodiments, the amide bond forming reaction proceeds with the acid chloride, a suitable base, and in a suitable solvent. In some embodiments, the suitable base is N-methylmorpholine, triethylamine, diisopropylethylamine, sec-butylamine, 1,2,2,6,6-pentamethylpiperidine, tributylamine, and 1,8-diazabicyclo[5.4.0]undec-7-ene (DBU), or the like. In some embodiments, the suitable base is triethylamine. In some embodiments, the suitable solvent is acetonitrile, dimethylformamide, diethyl ether, ethanol, tetrahydrofuran, isopropyl alcohol, 1,4-dioxane, toluene, or a combination thereof. In some embodiments, the suitable solvent is toluene.

In some embodiments, the reaction of Step 3 is performed at an elevated temperature. In some embodiments, the reaction of Step 3 is performed at a temperature of from about 50° C. to about 60° C. In some embodiments, the reaction of Step 3 is performed at ambient temperature.

In some embodiments, the compound of Formula 5 is the hydrochloride salt of methyl 2-amino-2,3-dihydro-1H-indene-2-carboxylate (Compound 5a):

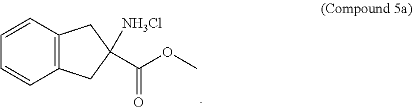

In some embodiments, the compound of Formula 6, is Compound 6a:

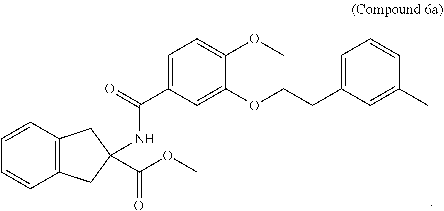 [SMILES: COC(=O)C1(NC(=O)c2ccc(OC)c(OCCc3cccc(C)c3)c2)Cc2ccccc2C1]

### Step 4: Synthesis of a Compound of Formula 7 (Saponification)

In some embodiments, a compound of Formula 6 undergoes a saponification reaction to yield a compound of Formula 7. In some embodiments, the saponification proceeds by contacting the compound of Formula 6 with a hydroxide reagent having the formula M-OH in a suitable solvent to provide a compound of Formula 7.

In some embodiments, the hydroxide reagent is NaOH, KOH, or LiOH. In some embodiments, the hydroxide reagent is NaOH or KOH. In some embodiments, the hydroxide reagent is NaOH; and M⁺ is Na⁺. In some embodiments, the hydroxide reagent is provided as an aqueous solution. In some embodiments, the hydroxide reagent is about 0.1 M, about 0.5 M, about 1.0 M, about 2.0 M, about 5.0 M, about 10 M, or concentrated aqueous sodium hydroxide. In some embodiments, the hydroxide reagent is about 1.0 M aqueous sodium hydroxide.

In some embodiments, the suitable solvent for the saponification reaction is tetrahydrofuran, methanol, ethanol, ethylene glycol, acetonitrile, water, or a combination thereof. In some embodiments, the suitable solvent is a mixture of methanol and water.

In some embodiments, the saponification step is performed at an elevated temperature. In some embodiments, the saponification step is performed at a temperature of about 50° C. to about 70° C. In some embodiments, the saponification step is performed at a temperature of about 60° C.

In some embodiments, the saponification step is performed for at least 1 hour, at least 2 hours, at least 3 hours, or more. In some embodiments, the saponification step is performed for about 1 hour, about 2 hours, or about 3 hours. In some embodiments, the saponification step is performed for about 3 hours.

In some embodiments, the compound of Formula 7, is Compound 7a:

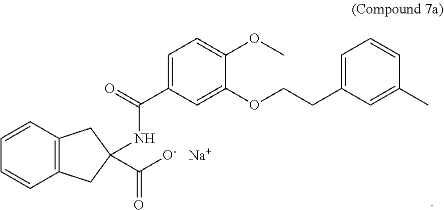 [SMILES: COc1ccc(C(=O)NC2(C(=O)[O-])Cc3ccccc3C2)cc1OCCc1cccc(C)c1 ; [Na+]]

In some embodiments, the compound of Formula 7 is not isolated prior to Step 5. In some such embodiments, Steps 4 and 5 are performed in the same reaction vessel. In some such embodiments, the reaction mixture of Step 4 is cooled to room temperature before proceeding to Step 5. In some such embodiments, the reaction mixture of Step 4 is cooled to room temperature before addition of the organic acid. In some such embodiments, the reaction mixture of Step 4 is cooled to 20° C. before addition of the organic acid.

### Step 5: Synthesis of Compound (Acidification)

In some embodiments, a salt of Formula 7 undergoes an acidification reaction to provide the free acid Compound I. In some embodiments, the acidification proceeds by contacting the compound of Formula 7 with a suitable acid in a suitable solvent to provide Compound I. In some embodiments, the acidification proceeds by contacting the compound of Formula 7 with a suitable organic acid in a suitable solvent to provide Compound I.

In some embodiments, the suitable solvent for the acidification reaction is tetrahydrofuran, methanol, ethanol, ethylene glycol, acetonitrile, water, or a combination thereof. In some embodiments, the suitable solvent is a mixture of methanol and water. In some embodiments, the compound of Formula 7 is not isolated from the saponification reaction, and the acidification reaction proceeds in the same vessel and in the same solvent as the saponification reaction.

In some embodiments, the acidification is performed with the use of a suitable organic acid. In some embodiments, the suitable organic acid is 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, isobutyric acid, lactic acid (DL), lactobionic acid, lauric acid, maleic acid, malic acid (-L), malonic acid, mandelic acid (DL), methanesulfonic acid, naphthalene-1,5-disulfonic acid, naphthalene-2-sulfonic acid, nicotinic acid, oleic acid, oxalic acid, palmitic acid, pamoic acid, phosphoric acid, proprionic acid, pyroglutamic acid (-L), salicylic acid, sebacic acid, stearic acid, succinic acid, tartaric acid (+L), thiocyanic acid, toluenesulfonic acid (p), or undecylenic acid. In some embodiments, the suitable organic acid is lactic acid, acetic acid, formic acid, citric acid, oxalic acid, malic acid, tartaric acid. In some embodiments, the suitable organic acid is citric acid. In some embodiments, the suitable organic acid is provided as an aqueous solution. In some embodiments, the suitable organic acid is about 0.1 M, about 0.5 M, about 1.0 M, about 1.5 M, or about 2.0 M aqueous citric acid. In some embodiments, the suitable organic acid is about 1.0 M aqueous citric acid.

In some embodiments, the pH of the solution after addition of the suitable organic acid is from about 6 to about 9. In some embodiments, the pH of the solution after addition of the suitable organic acid is from about 7 to about 8. In some embodiments, the pH of the solution after addition of the suitable organic acid is about 7.0, about 7.1, about 7.2, about 7.3, about 7.4, about 7.5, about 7.6, about 7.7, about 7.8, about 7.9, or about 8. In some embodiments, the pH of the solution after addition of the suitable organic acid is about 7.5.

### Crystallization

In some embodiments, Compound I is isolated and recrystallized.

In some embodiments, Compound I is crystallized directly from the reaction mixture.

In some embodiments, the reaction mixture is cooled to facilitate crystallization. In some embodiments, the reaction mixture is cooled to from about 0° C. to about 10° C. In some embodiments, the reaction mixture is cooled to about 10° C. In some embodiments, the reaction mixture is quickly cooled. In other embodiments, the reaction mixture is cooled slowly. In some embodiments, the reaction mixture is cooled over about 1 hour, 2 hours, 3 hours, 4 hours, or more. In some embodiments, the cooled mixture is maintained at the lower temperature for a period of about 1 hour, 2 hours, 3 hours, 4 hours, or more.

In some embodiments, the reaction mixture is cooled from 20° C. to 10° C. over a period of about 3 hours. In some such embodiments, the reaction mixture maintained at about ° C. for about 1 hour.

In some embodiments, the reaction mixture is seeded with pure crystalline Form 1 prior to cooling to facilitate crystallization. In some such embodiments, the reaction mixture is seeded with about 1% w/w, about 2% w/w, about 3% w/w, about 4% w/w, or about 5% w/w pure crystalline Form 1. In some such embodiments, the reaction mixture is seeded with about 2% w/w pure crystalline Form 1.

In some embodiments, Compound I is isolated as crystalline Form 1. In some embodiments, isolated Compound I is isolated as crystalline Form 1 and shows no evidence of other forms.

In some embodiments, Compound I is synthesized as outlined in Scheme 4.

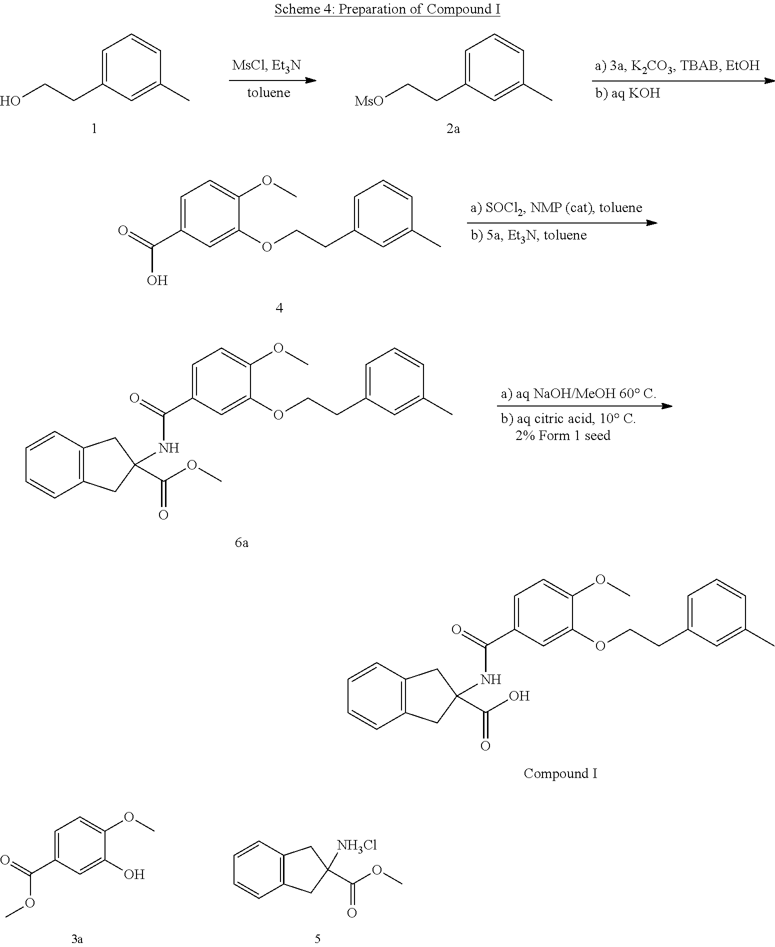

Briefly, in some embodiments, the compound of formula 1 is treated with MSCl and a suitable base (e.g., Et₃N) to yield compound 2a. In some embodiments, compound 2a is reacted with compound 3a, followed by saponification to yield compound 4. In some embodiments, acid compound 4 undergoes an amide bond formation reaction with compound 5a to yield compound 6a. In some embodiments, compound 6a undergoes a saponification reaction with a suitable hydroxide reagent (e.g., NaOH, KOH, or LiOH); and the resulting salt is acidified with a suitable organic acid to provide Compound I. In some embodiments, Compound I is crystallized as described herein.

In some embodiments, compounds and solid state forms described herein are synthesized as outlined in the Examples.

Described herein is a pharmaceutical composition of compound 2-(4-methoxy-3-(3-methylphenethoxy)benzamido)-2,3-dihydro-1H-indene-2-carboxylic acid (Compound I) substantially free of impurities. In some embodiments, the pharmaceutical composition is substantially free of Compound I impurities. In some embodiments, the pharmaceutical composition comprises less than about 1% w/w of Compound I impurities. In some embodiments, the pharmaceutical composition comprises less than about 1% w/w, less than about 0.75% w/w, less than about 0.50% w/w, less than about 0.25% w/w, less than about 0.20% w/w, less than about 0.15% w/w, less than about 0.10% w/w, or less than about 0.05% w/w of Compound I impurities. In some embodiments, the amount of Compound I impurities is undetectable. In some embodiments, the amount of Compound I impurities is undetectable by NMR, HPLC, or the like.

In some embodiments, the Compound I impurities comprise one or more degradants of Compound I. In some embodiments, the Compound I impurities comprise one or more intermediates used in the synthesis of Compound I. In some embodiments, the Compound I impurities are selected from:

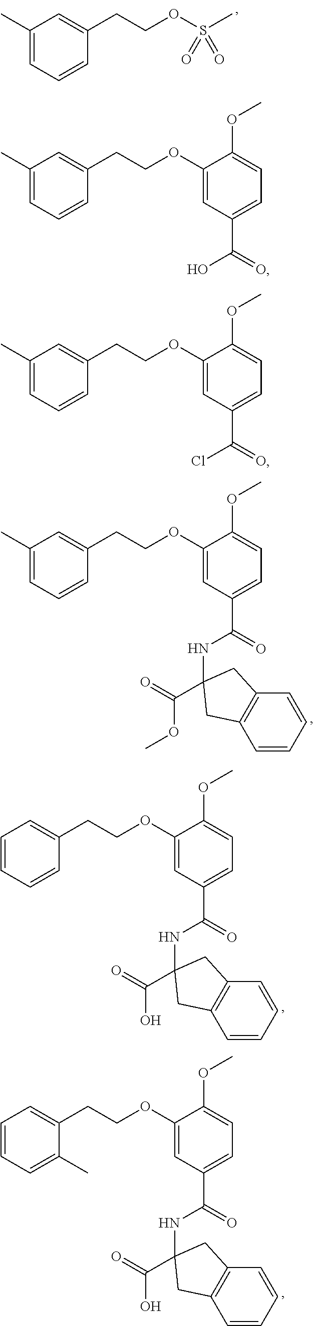

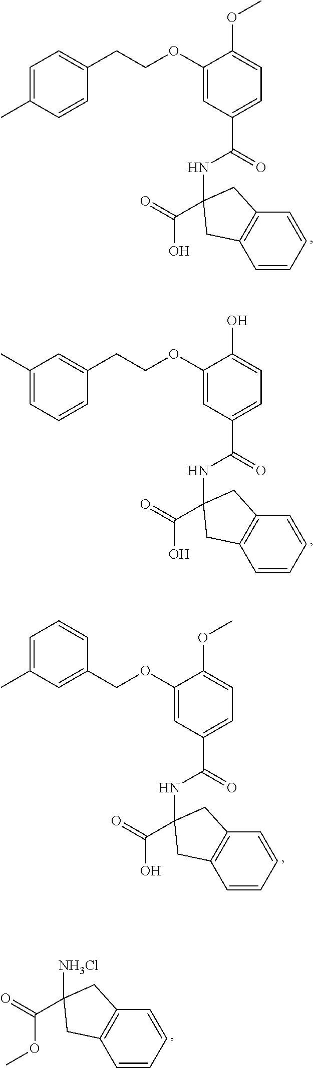

or a combination thereof.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts of Compound I are obtained by reacting Compound I with a base. In some embodiments, the base is an inorganic base. In such situations, the acidic proton of Compound I is replaced by a metal ion, e.g., lithium, sodium, potassium, magnesium, or calcium. Acceptable inorganic bases used to form salts with Compound I include, but are not limited to, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, or magnesium salt. In some embodiments, described herein is the sodium salt of Compound I.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound I, comprise an organic solvent(s). In some embodiments, compositions comprising Compound I include a residual amount of an organic solvent(s). In some embodiments, compositions comprising Compound I comprise a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

In some embodiments, the compositions comprising Compound I include a detectable amount of an organic solvent. In some embodiments, the organic solvent is a Class 3 solvent.

In other embodiments are compositions comprising Compound I wherein the composition comprises a detectable amount of solvent that is less than about 1%, wherein the solvent is selected from acetone, 1,2-dimethoxyethane, acetonitrile, ethyl acetate, tetrahydrofuran, methanol, ethanol, heptane, and 2-propanol. In a further embodiment are compositions comprising Compound I wherein the composition comprises a detectable amount of solvent which is less than about 5000 ppm. In yet a further embodiment are compositions comprising Compound I, wherein the detectable amount of solvent is less than about 5000 ppm, less than about 4000 ppm, less than about 3000 ppm, less than about 2000 ppm, less than about 1000 ppm, less than about 500 ppm, or less than about 100 ppm.

The methods and formulations described herein include the use of N-oxides (if appropriate), or pharmaceutically acceptable salts of compounds having the structure disclosed herein, as well as active metabolites of these compounds having the same type of activity.

In some embodiments, sites on the organic radicals (e.g. alkyl groups, aromatic rings) of compounds disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or altered metabolic pathways to reduce undesirable metabolites or reduced dosage requirements.

In some embodiments, one or more hydrogen atoms on Compound I are replaced with deuterium. In some embodiments, substitution with deuterium affords certain therapeutic advantages resulting from greater metabolic stability, such as, for example, increased in vivo half-life or reduced dosage requirements.

In one aspect, described is a compound with the following structure:

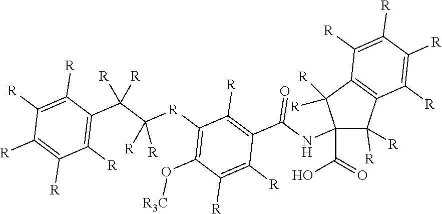

wherein,

each R is independently selected from hydrogen or deuterium,

or a pharmaceutically acceptable salt thereof.

In some embodiments, the compounds disclosed herein possess one or more stereocenters and each stereocenter exists independently in either the R or S configuration. For example, in some embodiments, the compound disclosed herein exists in the R configuration when one stereocenter is present. In other embodiments, the compound disclosed herein exists in the S configuration when one stereocenter is present. In some embodiments, the compound disclosed herein exists in the RR configuration when two stereocenters are present. In some embodiments, the compound disclosed herein exists in the RS configuration when two stereocenters are present. In some embodiments, the compound disclosed herein exists in the SS configuration when two stereocenters are present. In some embodiments, the compound disclosed herein exists in the SR configuration when two stereocenters are present.

The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers of compounds disclosed herein is carried out using covalent diastereomeric derivatives of the compounds described herein. In another embodiment, diastereomers of compounds disclosed herein are separated by separation/resolution techniques based upon differences in solubility. In other embodiments, separation of stereoisomers of compounds disclosed herein is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

Separation of individual enantiomers from a racemic mixture is possible by the use of chiral supercritical fluid chromatography (SFC) or chiral high performance liquid chromatography (HPLC). In some embodiments, enantiomers described herein are separated from each other by the use of chiral SFC or chiral HPLC. In some embodiments, compounds disclosed herein that include one or more chiral centers (e.g. compounds disclosed herein that include the moiety trans-octahydro-1H-pyrido[3,4-b]morpholin-6-yl) are separated into individual enantiomers using chiral SFC or chiral HPLC. A wide variety of conditions and suitable columns are available.

Daicel polysaccharide chiral stationary phases (CSPs) are among the columns used for chiral SFC separations. In some embodiments, Daicel analytical immobilized and coated CHIRALPAK and CHIRALCEL HPLC columns can be used for SFC analysis.

In some embodiments, screening for the suitability of using a SFC column is performed on the four main immobilized phases (CHIRALPAK IA, IB, IC and ID) and the four main coated columns (CHIRALPAK AD and AS and CHIRALCEL OD and OJ), with varying concentrations of organic modifier. A variety of column phases are available, including but not limited to OD and OJ, OX and OZ chlorinated phases, and a range of complementary cellulose based CHIRALCEL phases including OA, OB, OC, OF, OG and OK.

Non-limiting examples of chiral selectors contemplated for use in the separation of enantiomers include amylose tris (3, 5-dimethylphenylcarbamate), cellulose tris (3, 5-dimethylphenylcarbamate), cellulose tris (3, 5-dichlorophenylcarbamate), amylose tris (3-chlorophenylcarbamate), amylose tris (3, 5-dichlorophenylcarbamate), amylose tris (3-chloro, 4-methylphenylcarbamate), amylose tris ((S)-alpha-methylbenzylcarbamate), amylose tris (5-chloro-2-methylphenylcarbamate), cellulose tris (4-methylbenzoate), cellulose tris (4-chloro-3-methylphenylcarbamate), and cellulose tris (3-chloro-4-methylphenylcarbamate).

Non-limiting examples of chiral columns contemplated for use in the separation of enantiomers include CHIRALPAK IA SFC, CHIRALPAK AD-H SFC, CHIRALPAK D3 SFC, CHIRALCEL OD-H SFC, CHIRALPAK IC SFC, CHIRALPAK ID SFC, CHIRALPAK IE SFC, CHIRALPAK IF SFC, CHIRALPAK AZ-H SFC, CHIRALPAK AS-H SFC, CHIRALPAK AY-H SFC, CHIRALCEL OJ-H SFC, CHIRALCEL OX-H SFC, and CHIRALCEL OZ-H SFC.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “halo” or, alternatively, “halogen” or “halide” means fluoro, chloro, bromo or iodo. In some embodiments, halo is fluoro, chloro, or bromo.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds disclosed herein, or a pharmaceutically acceptable salt thereof, are used in the preparation of medicaments for the treatment of diseases or conditions in a mammal that would benefit from modulation of LPA₁ receptor activity. Methods for treating any of the diseases or conditions described herein in a mammal in need of such treatment, involves administration of pharmaceutical compositions that include at least one compound disclosed herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound disclosed herein, or a pharmaceutically acceptable salt thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In any of the aforementioned aspects are further embodiments in which the effective amount of the compound disclosed herein, or a pharmaceutically acceptable salt thereof, is: (a) systemically administered to the mammal; and/or (b) administered orally to the mammal.

In some embodiments, compound I, or a pharmaceutically acceptable salt thereof, is administered is dose selected from about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, and about 400 mg. In some embodiments, the dose is administered once a day. In some embodiments, the dose is administered twice a day.

### Articles of Manufacture and Kits

Disclosed herein, in certain embodiments, are kits and articles of manufacture for use with one or more methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include one or more of the compounds described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

### Abbreviations

Aq or aq: aqueous;

CN or MeCN: acetonitrile;

DCM: dichloromethane;

DSC: differential scanning calorimetry;

DVS: dynamic vapor sorption;

Et: ethyl;

EtOAc: ethyl acetate;

EtOH: ethanol;

equiv or eq.: equivalents;

FTiR: Fourier transform infrared

h or hr: hour;

hrs: hours;

high-performance liquid chromatography;

LC-MS or LCMS or LC/MS: liquid chromatography-mass spectrometry;

M: molar;

Mf K: methyl ethyl ketone;

Me: methyl;

MeOH: methanol;

Me-THF or methyl THF: 2-methyltetrahydrofuran;

mins or min: minutes;

NaOH: sodium hydroxide;

NMR: nuclear magnetic resonance;

RH: relative humidity;

rt or RT: room tempera pure;

SCXRD: single crystal x-ray diffraction;

ssNMR: solid state nuclear magnetic resonance;

TGA: thermogravimetric analysis;

THF: tetrahydrofuran,

vol: volume, typically used for reaction volume or ratio of solvents;

w/w: weight ratio; and

XRPD: X-ray powder diffraction.

The following examples are provided for illustrative purposes only and not to limit the scope of the claims provided herein.

### Example 1: Preparation of 2-(4-methoxy-3-(3-methylphenethoxy)benzamido)-2,3-dihydro-1H-indene-2-carboxylic Acid (Compound I)

The preparation of Compound I has been previously described (see, WO 2009/135590, U.S. Pat. Nos. 8,362,073, 8,445,530, 8,802,720, 9,328,071, each of which is incorporated by reference in its entirety).

Previously described preparations of Compound I provided Form 2.

### Example 1a: Preparation of 2-(4-methoxy-3-(3-methylphenethoxy)benzamido)-2,3-dihydro-1H-indene-2-carboxylic Acid (Compound I, Form 1)

Compound I (Form 2) was suspended in THF (a minimal amount of THF was used (5 v/w)) and stirred at about 22° C. for about 5 to about 7 days. The vessel or cake was not washed with any further solvent. Compound I (Form 1) was obtained. Coversion of Form 2 to Form 1 did not occur for about two to four days.

### Example 1a: Alternative preparation of 2-(4-methoxy-3-(3-methylphenethoxy)benzamido)-2,3-dihydro-1H-indene-2-carboxylic Acid (Compound I, Form 1)

An alternative preparation of Compound I is described here.

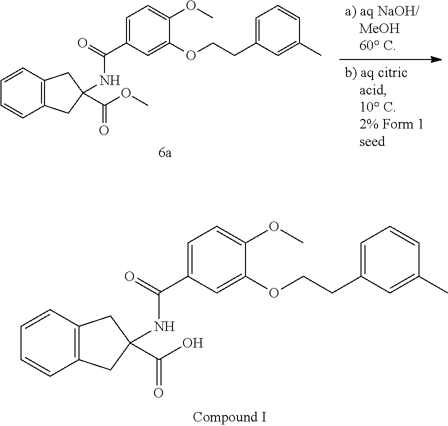 [SMILES: COC(=O)C1(NC(=O)c2ccc(OC)c(OCCc3cccc(C)c3)c2)Cc2ccccc2C1 ; COc1ccc(C(=O)NC2(C(=O)O)Cc3ccccc3C2)cc1OCCc1cccc(C)c1]

a) Saponification: Methyl 2-(4-methoxy-3-(3-methylphenethoxy)benzamido)-2,3-dihydro-1H-indene-2-carboxylate (6a, 10 g, 22 mmol, 1 eq) was dissolved in methanol (164 mL, 1.64 vol) and was heated to 50° C. with stirring. Aqueous NaOH (1 M, 26 mL, 1.21 eq) was added to the stirred solution over 30 min followed by water (3 mL, 0.3 vol). The reaction was stirred at 60° C. for 3 h, at which point LCMS showed complete reaction of 6a. The reaction mixture was cooled to 20° C. and filtered to remove insoluble material. The pH of the resultant solution was 13.2.

b) Acidification/Crystallization: The solution was acidified with 1 M citric acid (aq) to pH 7.5. The solution was seeded with crystals of Form 1 (2% by mass), cooled to 10° C. over 3 hrs, and was kept at 10° C. for 1 hr. The resulting suspension was filtered and solids were washed with 1:1 water:methanol (2×5 vol) followed by methanol (2×5 vol). The solid was dried in a vacuum oven at 40° C. to yield Compound I (9.2 g, 95%, Form 1 by XRPD).

### Example 2: Preparation of Solid State Forms—Evaporation from Solvent at Room Temperature

Compound I was dissolved in a variety of solvents at room temperature (about 25° C.) to provide solutions of Compound I with a maximum concentration of 10 mg/mL. The input material in these experiments was a mixture of polymorphic forms 1, 2, and 3.

The maximum concentration used in this set of experiments was 10 mg/mL. Solubility was highest in THF at >10 mg/mL. A solubility of 4-6 mg/mL was observed in acetone and MEK, while in methanol about 2-3 mg/mL was observed. The solubility was estimated at less than 2 mg/mL for: 1-butanol, butyl acetate, hexane, ethanol, ethyl acetate, isobutyl alcohol, 1-pentanol, isopropanol, acetonitrile, dichloromethane, chloroform, and water.

When the observed solubility was greater than 2 mg/mL, the solutions were filtered and evaporated at 25° C. to isolate the solid.

The crystal form determinations for each individual sample are listed in the following table:

| Solvent | Form (XRPD) |
|---|---|
| Acetone | Form 1 |
| Methyl Ethyl Ketone (MEK) | Form 1 |
| THF | Form 1 |
| Methanol | Form 1 + Form 2 |

Samples that were prepared by evaporation from solvents with lower to intermediate polarity (Acetone, MEK, THF) showed the presence of pure Form 1. The sample prepared from a solvent with higher polarity (methanol) showed the presence of both Form 1 and Form 2. The correlation of the presence of Form 2 with solvents of higher polarity was a consistent observation with other methods of isolation.

TGA results for all samples showed less than 1.0% weight loss up to 200° C.

### Acetone: Form 1

No evidence of other polymorphs were observed. The DSC scan shows three endotherms between 190-220° C. The first endotherm, at approximately 192-197° C., is attributed to the transformation of Form 1. The second endotherm is consistent with the melting of Form 3, followed by recrystallization and melting of Form 2 (onset 214° C.).

### Methyl Ethyl Ketone (MEK): Form 1

No evidence of other polymorphs was observed. The DSC scan shows the initiation of an endotherm at approximately 190-195° C. followed by recrystallization and melting of Form 2 at approximately 213° C.

### THF: Form 1

No evidence of other polymorphs was observed. The DSC scan shows the transformation of Form 1 at approximately 190° C. followed by multiple exothermic events (recrystallization) followed by melting of Form 2 at approximately 213° C.

### Methanol: Form 1+Form 2

The XRPD shows evidence of both Form 1 and Form 2 at 5-6° and 8.5-9.5° (2-theta). The DSC scan shows only a single endotherm consistent with the melting point of Form 2.

### Example 3: Preparation of Solid State Forms—Evaporation from Solvents at Elevated Temperature

Compound I was dissolved in a variety of solvents at elevated temperature (approximately at solvent boiling point) to provide solutions of Compound I with a maximum concentration of 15 mg/mL. The input material in these experiments was a mixture of polymorphic forms 1, 2, and 3.

When concentrations of Compound I was greater than 2 mg/mL, the solutions were filtered and evaporated at 25° C. to isolate the solid.

The crystal form determinations and estimated hot solubilities for each individual sample are listed in the following table:

| Solvent | Hot Solubility (est) | | Form |
|---|---|---|---|
| Acetone | >10 | mg/mL | Form 1 |
| 1-Butanol | >10 | mg/mL | Form 2 |
| Butyl Acetate | ~10 | mg/mL | Form 2 |
| Hexane | <2 | mg/mL | n.d. |
| Ethanol | >10 | mg/mL | Form 2 |
| Ethyl Acetate | 5-10 | mg/mL | Form 2 predominant |
| Methyl Ethyl Ketone (MEK) | >15 | mg/mL | Form 1 |
| Isobutyl Alcohol | >10 | mg/mL | Form 1 + Form 2 |
| 1-Pentanol | >10 | mg/mL | Form 1 + Form 2 |
| 2-Propanol | 7-10 | mg/mL | Form 2 + Form 3 |
| Acetonitrile | 7-10 | mg/mL | Form 2 |
| Dichloromethane (DCM) | <4 | mg/mL | n/a (oil) |
| Methanol | >10 | mg/mL | Form 2 |
| Chloroform | 4-5 | mg/mL | Form 3 |
| Water | <2 | mg/mL | n.d. |

n.d. = not determined

The solvents of higher polarity (methanol, ethanol, acetonitrile) were more likely to produce Form 2. Intermediate polarity solvents (Acetone, MEK) were more likely to produce Form 1. Mixtures of Form 2 and Form 1 were observed in ethyl acetate, 1-pentanol, and isobutyl alcohol. Pure Form 3 was observed only in chloroform. The experiment in dichloromethane produced an oil.

TGA results for all samples showed less than 1.0% weight loss up to 200° C., with the exception of ethyl acetate (1.5%).

### Acetone: Form 1

No evidence of additional forms was observed. The DSC scan shows a weak endotherm that initiated at approximately 190° C., followed by an additional endotherm/exotherm at 200-205° C. and finally an endotherm at approximately 214° C.

n-Butanol: Form 2

There was no evidence of additional forms by XRPD. The DSC scan shows a single endotherm at 214° C., consistent with the melting point for Form 2.

### Butyl Acetate: Form 2

DSC data shows a single endotherm with a melting point (onset approximately 215° C.) consistent with the presence of Form 2.

### Ethanol: Form 2

No evidence of other forms was observed. The DSC scan shows a single melting peak at 215° C. consistent with presence of Form 2.

### Ethyl Acetate: Form 2 (Predominant)+Possible Form 1

The XRPD pattern appears to be similar to the reference pattern for Form 2, however a shoulder is observed between 5-6 degrees 2-theta. The location of the shoulder is consistent with the presence of Form 1. The DSC scan shows a single melting peak at approximately 213° C.

### Methyl Ethyl Ketone (MEK): Form 1

No evidence of other forms was observed. The DSC scan shows multiple endotherms as Form 1 appears to melt/transform to Form 3 (Form 3 melting at approximately 205° C.). A third endotherm is observed at 213° C. indicating that the sample has transformed to Form 2.

### Isobutyl Alcohol: Form 1+Form 2

The XRPD data show evidence of both Form 1 and Form 2 at 5-6 degrees and 8.5-9.5 degrees 2-theta. The DSC scan shows a single endotherm at approximately 214° C.

### 1-Pentanol: Form 1+Form 2

The XRPD pattern shows evidence of both Form 1 and Form 2 at 5-6 degrees and 8.5-9.5 degrees 2-theta. The DSC scan shows a single endotherm at approximately 214° C.

### 2-Propanol: Form 2+Form 3

The XRPD shows a pattern consistent with predominantly Form 2 with slight evidence of Form 3 at 4.2 degrees 2-theta. The DSC scan shows a single endotherm at approximately 214° C.

### Acetonitrile: Form 2

The XRPD pattern is consistent with Form 2 with no evidence of other forms. The DSC scan shows a single endotherm at approximately 215° C.

### Methanol: Form 2

The XRPD pattern is consistent with Form 2 with no evidence of other forms. The DSC scan shows a single endotherm at approximately 214° C.

### Chloroform: Form 3

The XRPD pattern shows some similarity to that of Form 3, however a positive identity required additional characterization. FTIR was used to confirm the presence of Form 3.

### Example 4: Preparation of Solid State Forms—Conventional Recrystallization at 25° C. (Slow Cooling)

For recrystallization, both slow cooling (at 25° C.) and fast cooling (quench cooling to 0° C.) were utilized to attempt to generate new forms. Hot solutions (see Example 3, Elevated Temperature Evaporations) were cooled to 25° C. and the resultant collected solids were analyzed by XRPD.

The crystal form determinations for each individual sample isolated from slow cooling are listed in the following table:

| Solvent | Form |
|---|---|
| Acetone | Form 1 + Form 2 |
| 1-Butanol | Form 1 |
| Butyl Acetate | Form 1 + Trace |
| | Form 3 |
| Ethanol | Form 2 + Trace |
| | Form 1 |
| Ethyl Acetate | Form 1 + Form 2 |
| Methyl Ethyl Ketone (MEK) | Form 1 + Form 2 |
| Isobutyl Alcohol | Form 1 |
| 1-Pentanol | Form 1* |
| 2-Propanol | Form 1* |
| Acetonitrile | Form 1 + Form 2 |
| Methanol | Form 2 |
| Methyl THF | Form 1 |

*Possible form 2 polymorphic impurity

Predominantly Form 2 was observed from methanol and ethanol, the highest polarity solvents. Form 1 (pure or nearly pure) was observed most frequently, in particular from intermediate polarity solvents. These solvents included butyl acetate, isobutyl alcohol, 1-pentanol, 2-propanol, and methyl THF. Mixtures of Form 1 and Form 2 were observed from acetone, ethyl acetate, MEK, and acetonitrile.

TGA results for all samples showed less than 1.0% weight loss up to 200° C.

### Acetone: Form 1+Form 2

The XRPD pattern shows evidence of both Form 1 and Form 2 at 5-6 degrees (shoulder) and 8.5-9.5 degrees 2-theta. Characteristic reflections for Form 2 are evident at 7.2-8.2 degrees 2-theta. The DSC scan shows a weak endotherm that initiated at approximately 190° C., followed by an additional endotherm at approximately 215° C.

### 1-Butanol: Form 1

There was no evidence of additional forms by XRPD. The DSC scan shows an endotherm initiating at approximately 193-200° C. (characteristic of Form 1) followed by recrystallization and an endotherm consistent with the melting point for Form 2 at approximately 215° C.

### Butyl Acetate: Form 1+Trace Form 3

The XRPD pattern was consistent with Form 1, however there was a small peak suggesting a trace of Form 3 at approximately 4.3 degrees 2-theta. DSC data showed multiple events characteristic of Form 1 transformation (190-198° C.), Form 3 melting (200-205° C.), recrystallization and Form 2 melting (approximately 215° C.).

### Ethanol: Form 2+Trace Form 1

The XRPD pattern was consistent with Form 2. Possible trace evidence of Form 1 was observed at −5.2 degrees 2-theta. The DSC scan shows a single melting peak at 215° C.

### Ethyl Acetate: Form 1+Form 2

The XRPD pattern was shows evidence of both Form 1 and Form 2 at 5-6 degrees (shoulder) and 8.5-9.5 degrees 2-theta. Characteristic reflections for Form 2 are evident at 7.2-8.2 degrees 2-theta. The DSC scan shows a single endotherm at approximately 215° C.

### Methyl Ethyl Ketone (MEK): Form 1+Form 2

The XRPD pattern was shows evidence of both Form 1 and Form 2 at 5-6 degrees (shoulder) and 8.5-9.5 degrees 2-theta. Characteristic reflections for Form 2 are evident at 7.2-8.2 degrees 2-theta. The DSC scan shows a weak endotherm at 192-200° C., followed by an endotherm at approximately 216° C.

### Isobutyl Alcohol: Form 1

No evidence of other forms was observed. The DSC scan shows multiple endotherms corresponding to melting of Form 1 transformation (190-198° C.), melting of Form 3/transformation (200-204° C.), and melting of Form 2 (approximately 214° C.).

### 1-Pentanol: Form 1 (Predominant)

The XRPD pattern shows a pattern that is consistent with predominantly Form 1. A trace amount of Form 2 (reflection at ˜7.5 degrees 2-theta) may be present. The DSC scan shows multiple events at 195-205° C. associated with Form 1 melting/transformation and an endotherm at 215° C. consistent with melting of Form 2.

### 2-Propanol: Form 1 (Predominant)

The XRPD shows a pattern consistent with predominantly Form 1 with slight evidence of Form 2 at 7.4-7.5 degrees 2-theta. The DSC scan shows an endotherm at approximately 194-200° C., followed by an endotherm at 216° C.

### Acetonitrile: Form 1+Form 2

The XRPD shows evidence of both Form 1 and Form 2 at 5-6 degrees 2-theta. The DSC showed a single endotherm at 214° C.

### Methanol: Form 2

The XRPD pattern is consistent with Form 2. The DSC scan shows a single endotherm at approximately 216° C.

### Methyl THF: Form 1

There was no evidence of additional forms by XRPD. The DSC scan shows an endotherm between 194-198° C., followed by an endotherm at approximately 215° C.

### Example 5: Preparation of Solid State Forms—Conventional Recrystallization at 0° C. (Fast Cooling)

The crystal form determinations for each individual sample isolated from fast cooling at 0° C. are listed in the following table:

| Solvent | Form |
|---|---|
| Acetone | Form 1 |
| 1-Butanol | Form 1 |
| Butyl Acetate | Form 1 |
| Ethanol | Form 1 + Form 2 |
| Ethyl Acetate | Form 1 + Form 3 |
| Methyl Ethyl Ketone (MEK) | Form 1 |
| Isobutyl Alcohol | Form 4 * |
| 1-Pentanol | Form 1 |
| 2-Propanol | Form 4 * + Trace Form 3 |
| Acetonitrile | Form 1 + Form 2 + Form 3 |
| Methanol | Form 2 |
| Methyl THF | Form 1 |

* XRPD Pattern less defined than Form 4 reference

The isolation of Form 1 is most likely in solvents of lower to intermediate polarity. Predominantly Form 1 was isolated from acetone, 1-butanol, butyl acetate, MEK, 1-pentanol, and methyl THF. Predominantly Form 2 was isolated from methanol. XRPD data for samples isolated from isobutyl alcohol and 2-propanol appeared to resemble Form 4.

TGA results for all samples showed less than 1.0% weight loss up to 200° C.

### Acetone: Form 1

The XRPD pattern was consistent with Form 1. The DSC scan shows two weak endotherms at approximately 195-200° C. and 200-205° C., followed by an additional endotherm at approximately 216° C.

### 1-Butanol: Form 1

There was no evidence of additional forms by XRPD. The DSC scan shows multiple endotherms characteristic of Form 1 (194-200° C.), Form 3 melting/transformation (203-206° C.), and Form 2 melting (approximately 216° C.).

### Butyl Acetate: Form 1

There was no evidence of additional forms by XRPD. DSC data showed multiple events characteristic of Form 1 transformation (188-199° C.), Form 3 melting (203-204° C.), recrystallization, and Form 2 melting (approximately 216° C.).

### Ethanol—Form 1+Form 2

The XRPD pattern shows evidence of both Form 1 and Form 2 at 5-6 degrees and 8.5-9.5 degrees 2-theta. The DSC scan shows a weak endotherm at 195-200° C., followed by melting of Form 2 at approximately 215° C.

### Ethyl Acetate: Form 1+Form 3

The XRPD pattern was shows evidence of both Form 1 (5.3 degrees 2-theta) and Form 3 (4.2 degrees 2-theta). Characteristic reflections for Form 3 are evident at 6.5-7.5 degrees 2-theta. The DSC scan shows multiple endotherms characteristic of Form 1 transformation (195-199° C.), Form 3 melting/recrystallization (200-205° C.), and Form 2 melting (approximately 214° C.).

### Methyl Ethyl Ketone (MEK): Form 1

There was no evidence of additional forms by XRPD. The DSC scan shows a weak endotherm/exotherm at 195-200° C. and again at 202-204° C., and an endotherm at approximately 214° C.

### Isobutyl Alcohol: Form 4 (Predominant)

The XRPD pattern is similar to the pattern for Form 4, however the peaks are less defined. The DSC shows a weak exotherm at 150-160° C. followed by endotherms characteristic of Form 1 (190-198° C.), Form 3 (202-206° C.), and Form 2 respectively (214° C.).

### 1-Pentanol: Form 1

No evidence of other forms was observed. The DSC scan shows a broad endotherm at 190-198° C., a weak endotherm at 202-205° C., and an endotherm at approximately 214° C.

### 2-Propanol: Form 4+Form 3

The XRPD pattern shows similarity to Form 4 with evidence of some Form 3 (4.2 and ˜7 degrees 2-theta). The peaks appear less defined than Form 4. The DSC shows a weak exotherm at 140-160° C. Endotherms are observed at 188-195° C., 203-205° C., and 215° C.

### Acetonitrile: Form 1+Form 2+Form 3

The XRPD shows evidence of Forms 1, 2, and 3; there appeared to be only a trace level of Form 3. The DSC scan shows a weak endotherm and exotherm between 195-205° C., and an endotherm at approximately 215° C.

### Methanol: Form 2

The XRPD pattern is consistent with Form 2. A slight shoulder at 5.3 degrees 2-theta may indicate trace levels of Form 1. DSC shows a weak endotherm at approximately 165° C. followed by an endotherm at 215° C.

### Methyl THF: Form 1

The XRPD pattern is consistent with Form 1. The DSC scan shows the initiation of Form 1 melting at 198-199° C. followed by recrystallization and melting of Form 2 (approximately 214° C.).

### Example 6: Preparation of Solid State Forms—Isolation by Antisolvent Addition

Samples were isolated by crystallization via antisolvent by addition of a solution of THF (concentration 25 mg/mL, temperature 25° C.) to several antisolvents at a ratio of 1 to 4. Therefore, the final concentration of Compound I was 5 mg/mL. The crystal form determinations for each individual sample are listed in the following table:

| Solvent/Antisolvent | Form |
|---|---|
| THF/water | Form 1 |
| THF/acetonitrile | Form 2 |
| THF/ethyl acetate | Form 1 + Form 2 |
| THF/2-Propanol | Form 2 |
| THF/ethanol-water 1:1 | Form 2 |

Form 2 was observed when the antisolvent was acetonitrile, ethanol-water, and 2-propanol. Form 1 was observed when the weak solvent was water.

TGA results for all samples showed less than 1.0% weight loss up to 200° C.

### THF/Water: Form 1

The XRPD pattern was consistent with Form 1. The DSC scan showed multiple endotherms (195-198° C., 200-204° C.), followed by a melt at 215° C. The DSC was similar to previous Form 1 samples.

### THF/Acetonitrile: Form 2

The XRPD pattern was consistent with Form 2. The DSC scan showed a single endotherm at approximately 215° C.

### THF/Ethyl Acetate: Form 1+Form 2

The XRPD pattern shows evidence of both Form 1 and Form 2 at 5-6 degrees and 8.5-9.5 degrees 2-theta. The DSC scan shows a single endotherm at approximately 215° C.

### THF/2-Propanol: Form 2

The XRPD pattern was consistent with Form 2. The DSC scan showed a single endotherm at approximately 215° C.

### THF/Ethanol-Water (1:1): Form 2

The XRPD pattern was consistent with Form 2. The DSC scan showed a single endotherm at approximately 215° C.

### Example 7: Slurry Stability Studies

Slurry stability Maturation studies were initially conducted for the purpose of identifying the most relative stability of the crystal forms stable form at room temperature (25° C.).

In this set of experiments, both the individual forms and mixtures of forms were slurried in several solvents for 4 weeks, then filtered and analyzed to determine the resulting form. The first set of experiments included pure Form 1, Form 2, Form 3, and mixtures of these forms in equal amounts. The solvents included methanol, ethyl acetate, MEK, and methyl THF in order to investigate a range of solvent polarity. Additional experiments were conducted with a mixture of Form 1 and Form 4 in MEK and methanol.

Analysis was performed by FTIR due to ease of analysis for small quantities. It is interesting to note that the acid carbonyl is shifted to a different location for each polymorph. The results for the first set of experiments are listed below, comparing the starting form with the final form.

| | Starting Form | | | |
|---|---|---|---|---|
| | Form 1 | Form 2 | Form 3 | Forms 1, 2, 3 |
| Methanol-Final | Form 1 | Form 2 | Form 1 | Form 1 |
| MEK-Final | Form 1 | Form 1 | Form 1 | Form 1 |
| Methyl THF-Final | Form 1 | Form 2 | Form 1 | Form 1 |
| Ethyl Acetate-Final | Form 1 | Form 2 | Form 1 | NA |

Pure Form 1 was unchanged in each solvent, while pure Form 3 and mixtures of the three forms were observed to convert to Form 1. Form 2 was only observed to convert to Form 1 in MEK; no change was observed in methanol, methyl THF, or ethyl acetate.

The additional experiments (with Forms 1 and 4) showed that mixtures of Form 4 and Form 1 were observed to convert to Form 1 in both methanol and MEK. The data set clearly indicates that Form 1 is the most stable form at room temperature (25° C.); each of the other forms showed conversion in multiple experiments.

Slurry conversation studies were also conducted at 40-70° C. with Forms 1 and 2 to determine a transition temperature between the forms. A mixture of Form 1 and Form 2 at a (1:1 ratio) was slurried at 40° C., 50° C., 60° C., and 70° C. in two different solvents at each temperature and then analyzed to determine the direction of transformation. At 40° C. and 50° C., Methanol and MEK were used. At 60° C. and 70° C. MEK and 1-Pentanol were used. Results are summarized below.

| Solvent | 40° C. | 50° C. | 60° C. | 70° C. |
|---|---|---|---|---|
| Methanol | Form 1 | Form 1 | | |
| MEK | Form 1 | Form 1 | Form 2 | Form 2 |
| 1-Pentanol | | | Form 2 | Form 2 |

The data show transformation to Form 1 at 40-50° C. and transformation to Form 2 at temperatures of 60-70° C. This data set suggests that the Form 1/Form 2 transition temperature is between 50° C. and 60° C. and thus the two forms and enantiotropically related.

### Example 8: X-Ray Powder Diffraction (XRPD)

Although the following diffractometers were used, other types of diffractometers could be used. Furthermore, other wavelengths could be used and converted to the Cu Kα. In some embodiments, Synchrotron Radiation X-Ray Powder Diffraction (SR-XRPD) can be used to characterize the crystalline forms.

“Characteristic peaks”, to the extent they exist, are a subset of observed peaks and are used to differentiate one crystalline polymorph from another crystalline polymorph (polymorphs being crystalline forms having the same chemical composition). Characteristic peaks are determined by evaluating which observed peaks, if any, are present in one crystalline polymorph of a compound against all other known crystalline polymorphs of that compound to within ±0.2° 2-Theta.

### STOE Stadi-P Transmission Diffractometer

X-ray powder diffractions were performed with STOE Stadi-P transmission diffractometers using Cu-Kai radiation. Linear position sensitive detectors were used for capillary measurements and for samples in flat preparation, while image plate position sensitive detectors (IP-PSDs) were used for temperature-resolved XRPD, humidity-resolved XRPD and for robot samples in 96-well plates. The measured data was visualized and evaluated with the Software WinXPOW V2.12.

The 2-Theta peak values that are provided for the XRPD are within ±0.2° 2-Theta.

Characterization of Solid-State Forms of Compound I

The X-Ray powder diffraction pattern for crystalline Form 1 of Compound I is displayed in FIG. 1. The X-Ray powder diffraction pattern for crystalline Form 2 of Compound I is displayed in FIG. 6. The X-Ray powder diffraction pattern for crystalline Form 3 of Compound I is displayed in FIG. 10. The X-Ray powder diffraction pattern for crystalline Form 4 of Compound I is displayed in FIG. 13.

Characterization of Crystalline Form 1 of Compound I

The X-Ray powder diffraction pattern for crystalline Form 1 of Compound I is displayed in FIG. 1. Characteristic XRPD peaks include: 5.2±0.2° 2-Theta, 9.0±0.2° 2-Theta, 14.4±0.2° 2-Theta, and 17.7±0.2° 2-Theta.

Characterization of Crystalline Form 2 of Compound I

The X-Ray powder diffraction pattern for crystalline Form 2 of Compound I is displayed in FIG. 6. Characteristic XRPD peaks include: 5.6±0.2° 2-Theta, 7.6±0.2° 2-Theta, 8.1±0.2° 2-Theta, 9.4±0.2° 2-Theta, 14.9±0.2° 2-Theta, and 16.3±0.2° 2-Theta.

Characterization of Crystalline Form 3 of Compound I

The X-Ray powder diffraction pattern for crystalline Form 3 of Compound I is displayed in FIG. 10. Characteristic XRPD peaks include: 4.2±0.2° 2-Theta, 6.8±0.2° 2-Theta, 15.1±0.2° 2-Theta, 25.0±0.2° 2-Theta, 25.5±0.2° 2-Theta, and 26.4±0.2° 2-Theta.

In some embodiments, measurements on independently prepared samples on different instruments may lead to variability which is greater than ±0.2° 2-Theta. Independently prepared samples of crystalline Forms 1 and 2 were characterized on three additional diffractometers.

Malvern Panalytical Empyrean diffractometer

Instrument: Malvern Panalytical

Type: Empyrean with a Pixcel 1D Detector, a Copper XRD tube, a theta-theta goniometer and a sample changer.

Characterization of Crystalline Form 1 of Compound I

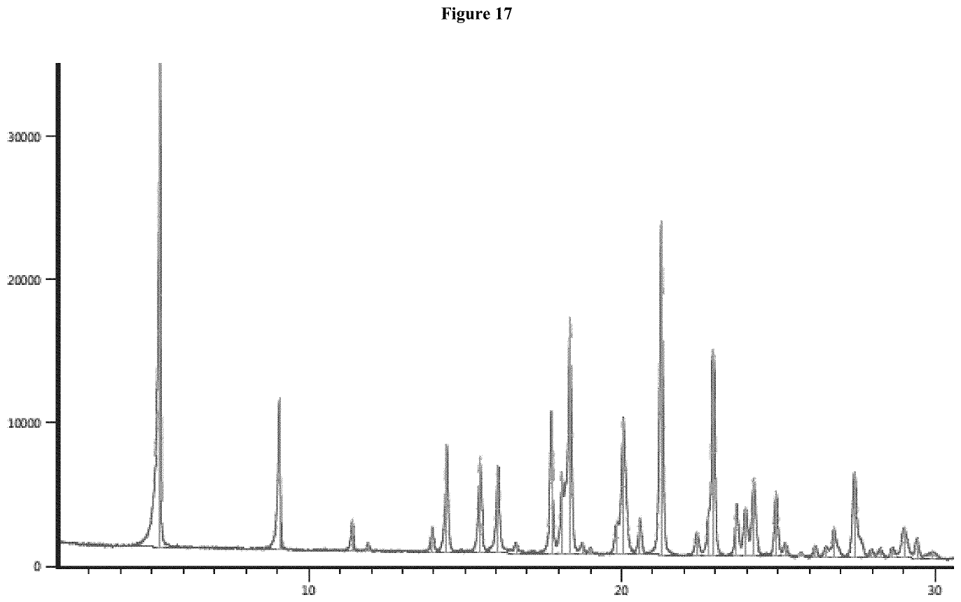

The X-Ray powder diffraction pattern for crystalline Form 1 of Compound I is displayed in FIG. 17. Characteristic XRPD peaks include: 5.2±0.2° 2-Theta, 9.0±0.2° 2-Theta, 14.4±0.2° 2-Theta, and 17.7±0.2° 2-Theta.

Characterization of Crystalline Form 2 of Compound I

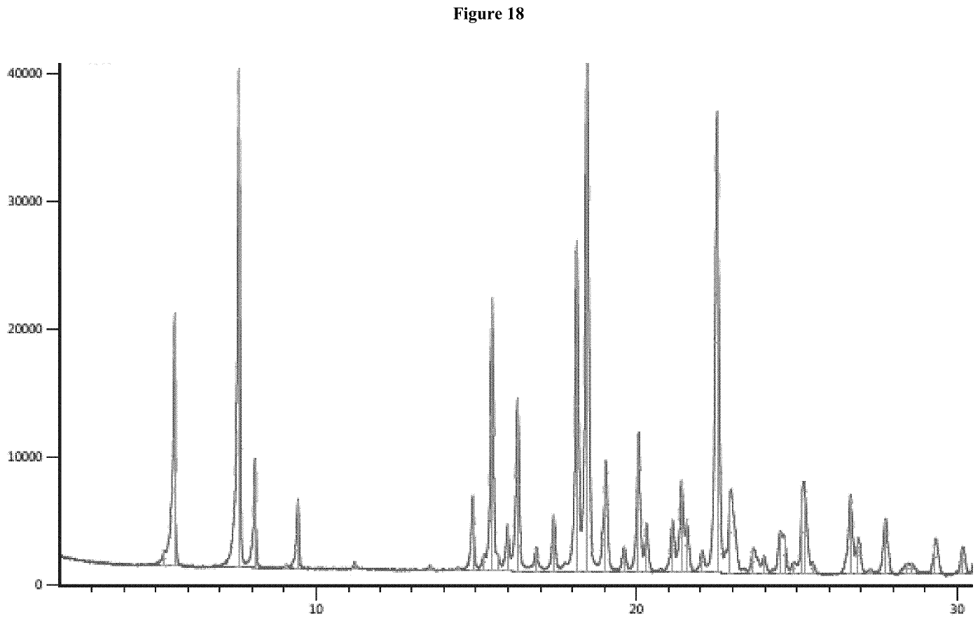

The X-Ray powder diffraction pattern for crystalline Form 2 of Compound I is displayed in FIG. 18. Characteristic XRPD peaks include: 5.6±0.2° 2-Theta, 7.6±0.2° 2-Theta, 8.1±0.2° 2-Theta, 9.4±0.2° 2-Theta, 14.8±0.2° 2-Theta, and 16.2±0.2° 2-Theta.

### Stoe Stadi P, G.52.SYS.S072

Equipment and Measurement Parameters

| | |
|---|---|
| Diffractometer: | Stoe Stadi P, G.52.SYS.S072 |
| Sample holders: | Stoe transmission sample holder, sample between |
| | two acetate foils with a 0.4 mm metal washer |
| | in between |
| Evaluation software: | WinXPOW by Stoe |

The X-ray diffraction pattern was recorded with
the following instrumental parameters:

| | |
|---|---|
| Radiation: | Cu Kα1; 40 kV, 40 mA |
| Collimator: | 0.5 × 10 mm |
| Detector: | Mythen1K |
| Detector distance: | resulting to 0.01°(2θ) intrinsic resolution |
| Monochromator: | Ge, curved monochromator |
| Sample rotation | 1 rps |
| Scan range: | at least 2-40°(2θ) |
| Step size: | 0.020°(2q) |
| Detector Step time: | 48 s |
| Detector step: | 1°(2q) |

Sample Preparation:

The cylindrical volume determined by the washer and the two sheets of foil was slightly overfilled with a small quantity of the sample and then smoothed with two glass slides to obtain a disk of powder. This specimen was then secured into a Ni-coated metal sample holder

Characterization of Crystalline Form 1 of Compound I

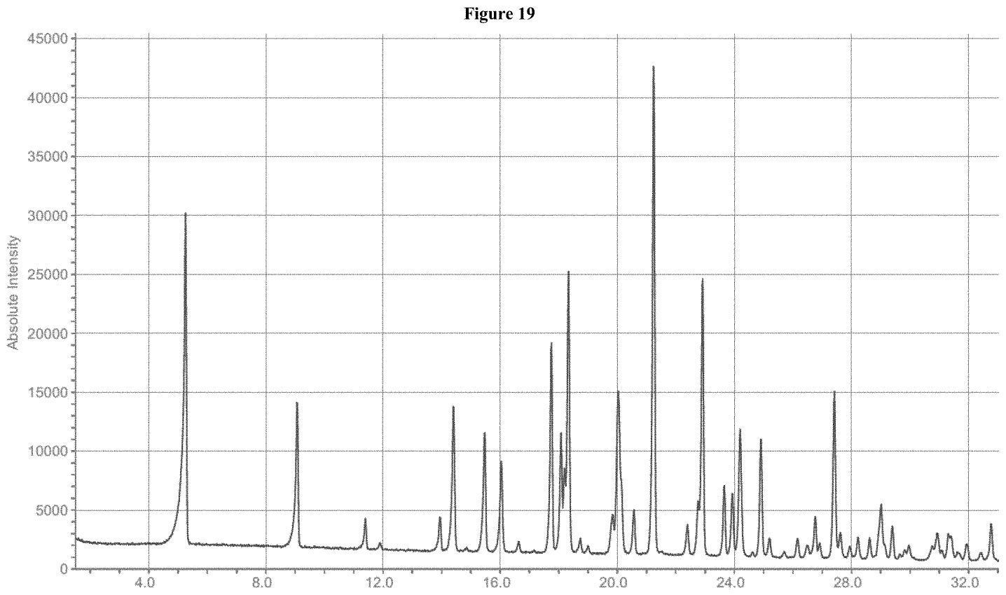

The X-Ray powder diffraction pattern for crystalline Form 1 of Compound I is displayed in FIG. 19. Characteristic XRPD peaks include: 5.2±0.2° 2-Theta, 9.0±0.2° 2-Theta, 14.4±0.2° 2-Theta, and 17.7±0.2° 2-Theta.

Characterization of Crystalline Form 2 of Compound I

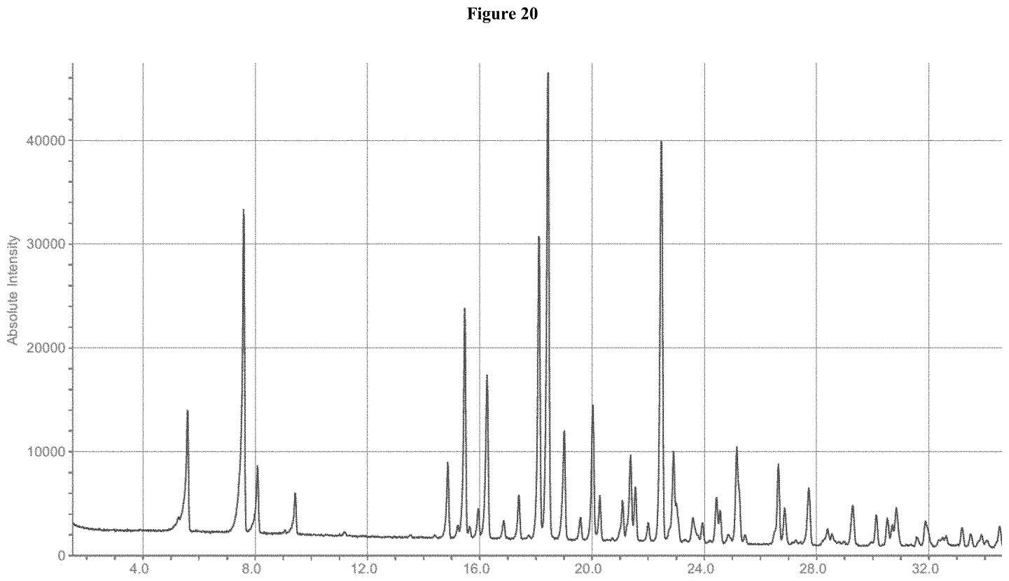

The X-Ray powder diffraction pattern for crystalline Form 2 of Compound I is displayed in FIG. 20. Characteristic XRPD peaks include: 5.5±0.2° 2-Theta, 7.5±0.2° 2-Theta, 8.0±0.2° 2-Theta, 9.4±0.2° 2-Theta, 14.8±0.2° 2-Theta, and 16.2±0.2° 2-Theta.

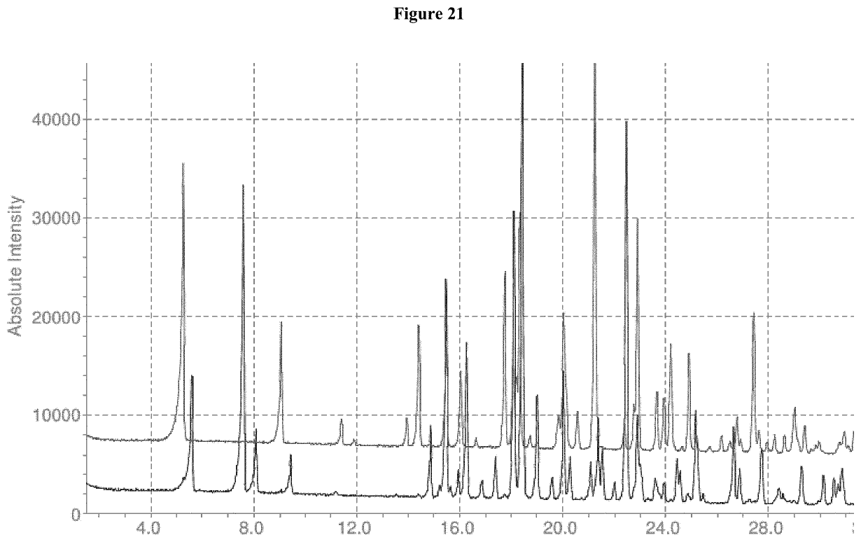

An overlay of the XRPD of Form 1 (top spectra) and Form 2 (bottom spectra) is displayed in FIG. 21.

### PANalytical X'Pert PRO MPD Diffractometer

X-Ray Powder Diffractometry (XRPD, Transmission Mode):

XRPD patterns were collected with a PANalytical X'Pert PRO MPD diffractometer using an incident beam of Cu radiation produced using an Optix long, fine-focus source. An elliptically graded multilayer mirror was used to focus Cu Kα X-rays through the specimen and onto the detector. A specimen of the sample was sandwiched between 3-μm-thick films and analyzed in transmission geometry. Prior to the analysis, a silicon specimen (NIST SRM 640f) was analyzed to verify the observed position of the Si 111 peak is consistent with the NIST-certified position. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5.

X-Ray Powder Diffraction Peak Identification Process:

Rounding algorithms were used to round each peak to the nearest 0.1° or 0.01° 20, depending upon the instrument used to collect the data and/or the inherent peak resolution. The location of the peaks along the x-axis (° 2-Theta) in both the figures and the tables were determined using TRIADS® v2.1.1 software and rounded to one or two significant figures after the decimal point based upon the above criteria. Peak position variabilities are given to within ±0.2° 2-Theta based upon recommendations outlined in the USP discussion of variability in x-ray powder diffraction (USP-NF 2021, Issue 2, <941>, Characterization of Crystalline and Partially Crystalline Solids by X-Ray Powder Diffraction (XRPD), 1_GUID-14EBB55E-0D24-45A1-A84F-FE4DCAAEE3E8_1_en-US, official prior to 2013). In some embodiments, measurements on independently prepared samples on different instruments may lead to variability which is greater than ±0.2° 2-Theta. For d-space listings, the wavelength used to calculate d-spacings was 1.5405929 Å, the Cu-Kα1 wavelength (Phys. Rev. A56(6) 4554-4568 (1997)).

Characterization of Crystalline Form 1 of Compound I

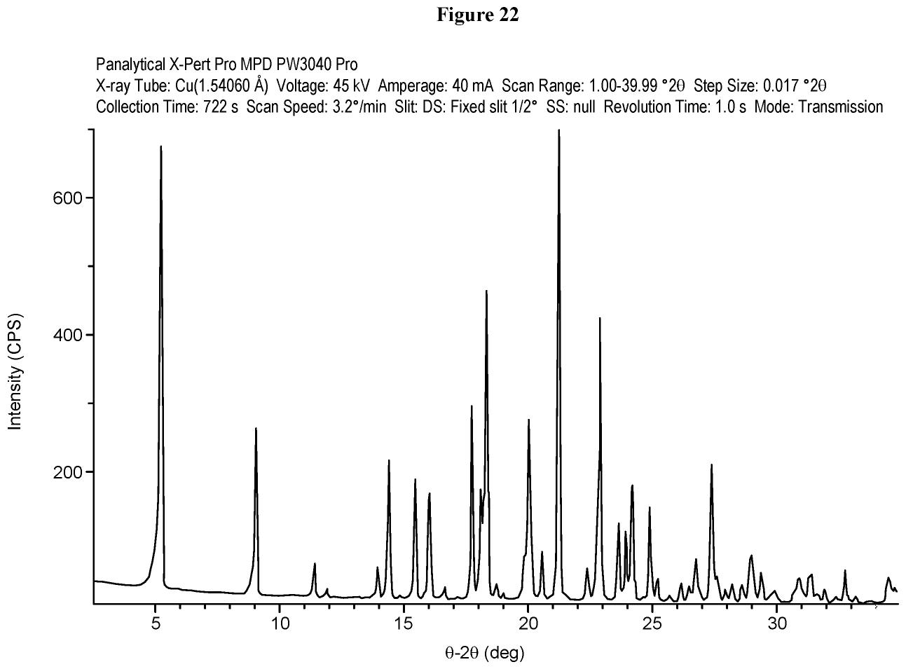

The X-Ray powder diffraction pattern for crystalline Form 1 of Compound I is displayed in FIG. 22. Characteristic XRPD peaks include: 5.2±0.2° 2-Theta, 9.0±0.2° 2-Theta, 14.4±0.2° 2-Theta, and 17.7±0.2° 2-Theta.

Characterization of Crystalline Form 2 of Compound I

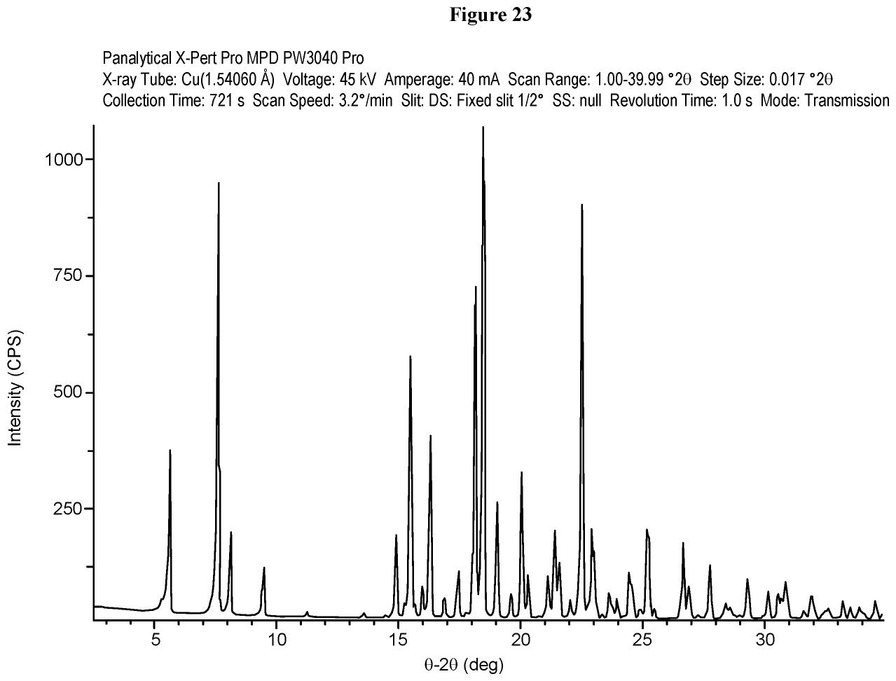

The X-Ray powder diffraction pattern for crystalline Form 2 of Compound I is displayed in FIG. 23. Characteristic XRPD peaks include: 5.5±0.2° 2-Theta, 7.5±0.2° 2-Theta, 8.0±0.2° 2-Theta, 9.4±0.2° 2-Theta, 14.8±0.2° 2-Theta, and 16.2±0.2° 2-Theta.

XRPD Limit Test Method with PANalytical X'Pert PRO MPD Diffractometer

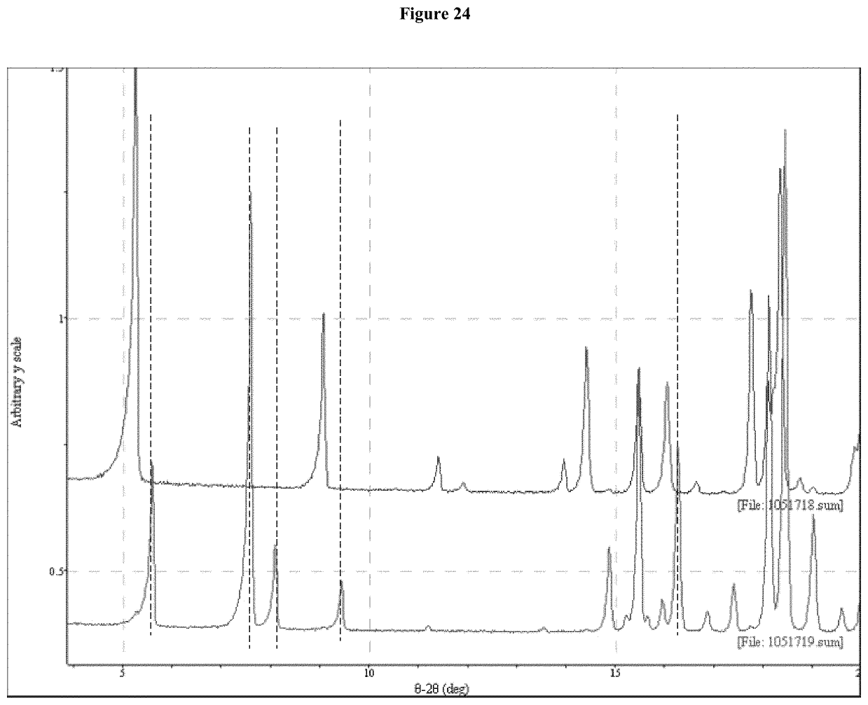

A non-limiting method development of an XRPD limit test for determining Form 2 in Form 1 drug substance is described. Specificity, the ability to unequivocally assess the analyte in the presence of components that may be expected to be present, was assessed by comparing XRPD patterns of forms 1 and 2. Specificity of Form 2 is good in the Form 1 drug substance as several peaks highlighted in FIG. 24 can be used for the quantification of Form 2 (bottom spectra) in Form 1 (top spectra).

Calibration Models Generation: Calibration standards containing 0-10% Form 2 in Form 1 were prepared by geometrically mixing components without any extra sample handling.

| | | | |
|---|---|---|---|
| Form 2 | Form 1 | Form 2 | Form 1 |

| | | |
|---|---|---|
| mg | % | XRPD |

| | | | | |
|---|---|---|---|---|
| 0.0000 | 100.0240 | 0.00 | 100.00 | 1054814 |
| 1.0260 | 98.9810 | 1.03 | 98.97 | 1054212 |
| 1.9830 | 98.0325 | 1.98 | 98.02 | 1054213 |
| 2.9730 | 96.9775 | 2.97 | 97.03 | 1054214 |
| 5.0255 | 94.9725 | 5.03 | 94.97 | 1054215 |
| 6.0170 | 93.9960 | 6.02 | 93.98 | 1054216 |
| 7.9800 | 92.0045 | 7.98 | 92.02 | 1054811 |
| 9.0410 | 90.9795 | 9.04 | 90.96 | 1054812 |
| 9.9990 | 90.0040 | 10.00 | 90.00 | 1054813 |

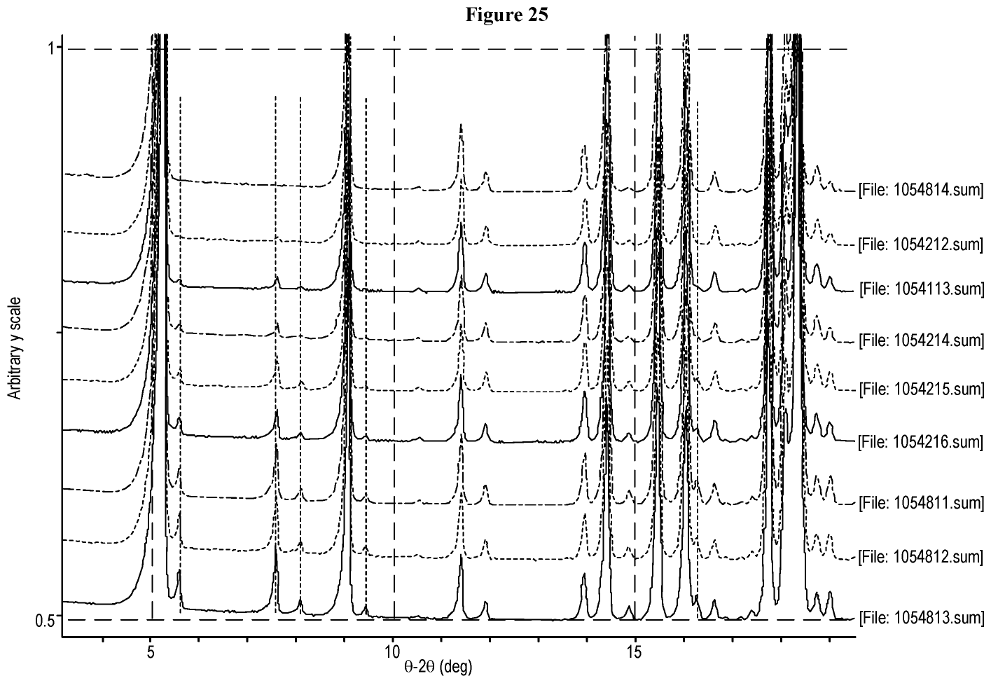

XRPD overlays of the calibration standards are shown in FIG. 25. Peaks unique to Form 2 were highlighted (with dotted lines) and showed good linearity based on visual assessment.

A spreadsheet was developed to calculate the areas of peaks approximately at 5.6°, 7.6°, and 8.1° which are normalized to the total peak area in the range of 4.0-25.5°.

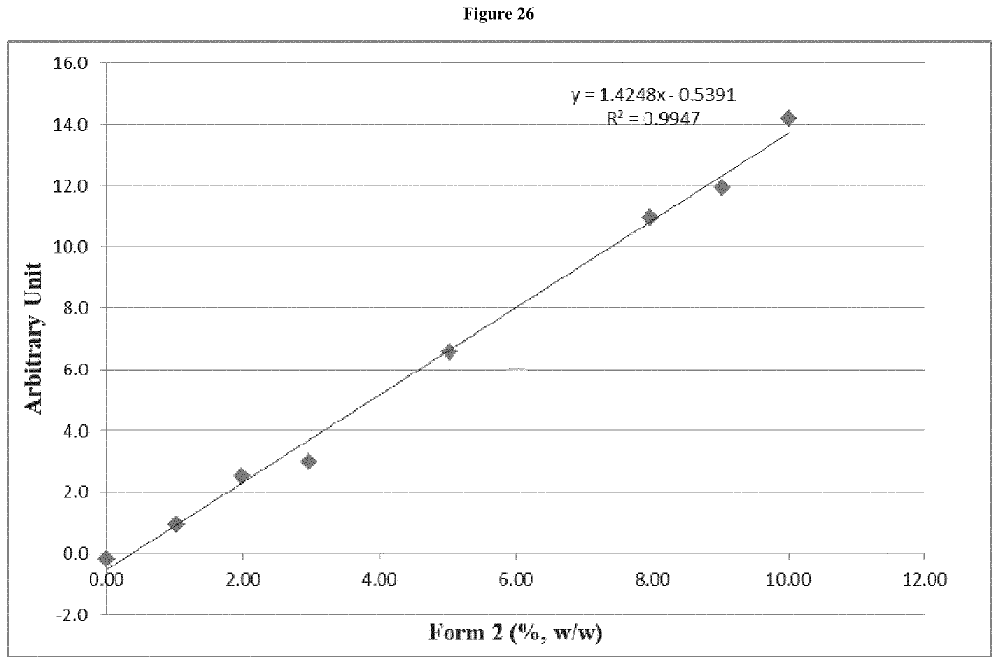

The calibration curve is shown in FIG. 26. Regression statistics along with the limit of detection (LOD) and limit of quantification (LOQ) are summarized below.

Regression Statistics

| | |
|---|---|
| Multiple R | 0.9974 |
| R Square | 0.9947 |
| Adjusted R Square | 0.9938 |
| Standard Error | 0.4316 |
| Observations | 8 |

ANOVA

| | df | SS | MS | F | Significance F |
|---|---|---|---|---|---|
| Regression | 1 | 210.54 | 210.54 | 1130.43 | 4.60825E−08 |
| Residual | 6 | 1.12 | 0.19 | | |
| Total | 7 | 211.66 | | | |

| | Coeffi- | Standard | | | Lower | Upper |
|---|---|---|---|---|---|---|
| | cients | Error | t Stat | P-value | 95% | 95% |
| Intercept | −0.54 | 0.25 | −2.13 | 0.08 | −1.16 | 0.08 |
| X Vari- | 1.42 | 0.04 | 33.62 | 4.6082E−08 | 1.32 | 1.53 |
| able 1 | | | | | | |

LOD and LOQ were calculated using the following equations:

LOD=(3.3×σ)/S

LOQ=(10×σ)/S

where σ is the standard error of linear regression and S is the slope of the calibration curve. The LOD and LOQ were calculated to be 1.0% and 2.8% (w/total), respectively.

### Example 9: Differential Scanning Calorimetry (DSC)

### 9.1 Mettler DSC822e

DSC measurements are performed with a METTLER DSC822e (module DSC822e/700/109/414935/0025). 40 μl Al-crucibles with sealed lid and pinhole are used. All measurements are carried out in a nitrogen gas flow of 50 mL/min and typical heating rate of 10° C./min. The measured data is evaluated via the software STARe V8.10.

### 9.2 Perkin Elmer Diamond DSC

DSC scans were obtained using a Perkin Elmer Diamond DSC. The samples were encapsulated in aluminum pans that were pierced to allow for residual solvent to be released. Scans were obtained at 10° C./min from 25-240° C. The system was calibrated with indium (MP 156.6° C.) and tin (MP 231.9° C.) prior to use.

### Characterization of Solid State Forms of Compound I

The DSC thermogram for crystalline Form 1 of Compound I is displayed in FIG. 2.

The DSC thermogram for crystalline Form 2 of Compound I is displayed in FIG. 7.

The DSC thermogram for crystalline Form 3 of Compound I is displayed in FIG. 11.

The DSC thermogram for crystalline Form 4 of Compound I is displayed in FIG. 14.

Differential Scanning calorimetry (DSC) thermogram thermal events for the solid state forms are as described in the following table:

| Solid State | |
|---|---|
| Form | DSC Thermal Events |
| Form 1 | three endothermic events having: an onset at about 198.5° C. |
| | and a peak at about 200.4° C.; an onset at about 204.8° C. |
| | and a peak at about 205.8° C.; and an onset at about |
| | 213.9° C. and a peak at about 216.3° C. |
| Form 2 | endothermic event having an onset at about 215.3° C. |
| | and a peak at about 216.4° C. |
| Form 3 | two endothermic events having: an onset at about 204.2° C. |
| | and a peak at about 205.3° C.; and an onset at about |
| | 213.6° C. and a peak at about 215.8° C. |

### Example 10: Thermogravimetric Analysis (TGA)

### Method 10.1: Mettler TGA851e

The thermogravimetric analyses are performed with a METTLER TGA851e (module TGA/SDTA851e/SF1100/042). 100 μl Al-crucibles with sealed lid and hole are used and the measurements are performed in a nitrogen gas flow of 50 mL/min. The measured data is evaluated via the software STARe V8.10.

### Method 10.2: Perkin Elmer Pyris System

TGA was obtained on either a Perkin Elmer Pyris System. The samples were run from 25-200° C. at 10° C./min. Accuracy of the system was verified using barium chloride dihydrate.

### Characterization of Solid State Forms of Compound I

The TGA pattern for crystalline Form 1 of Compound I is displayed in FIG. 3.

Thermogravimetric Analysis (TGA) patterns for the solid state forms are as described in the following table:

| Solid State | |
|---|---|
| Form | TGA Pattern |
| Form 1 | method 10.1: 15.4% w/w loss from about 287.9° C. to about |
| | 298.9° C.; |
| | method 10.2: TGA pattern (up to 200° C.) showed less than |
| | 1% weight loss |
| Form 2 | TGA pattern (up to 200° C.) showed less than 1% weight loss |
| Form 3 | TGA pattern (up to 200° C.) showed less than 1% weight loss |
| Form 4 | TGA pattern (up to 200° C.) showed less than 1% weight loss |

### Example 11: Dynamic Vapor Sorption (DVS)

Moisture sorption/desorption isotherms are recorded on a DVS-1 from SURFACE MEASUREMENT SYSTEMS. Two cycles are run at 25° C., in which the Relative Humidity (RH) is stepped from 0 to 95% and back to 0%. The data is evaluated with the software DVSWin V. 2.15.

Reversible water uptake for Form 1 of Compound I as determined by DVS is less than 1% (˜−0.1% w/w between 0 and 95% RH).

### Example 12: Fourier Transform Infrared (FTIR) Spectroscopy

Nicolet Magna 750 system was used to collect FTIR of the different solid state forms of Compound I. Samples were prepared at 1% concentration in KBr and compressed at 10,000 lbs.

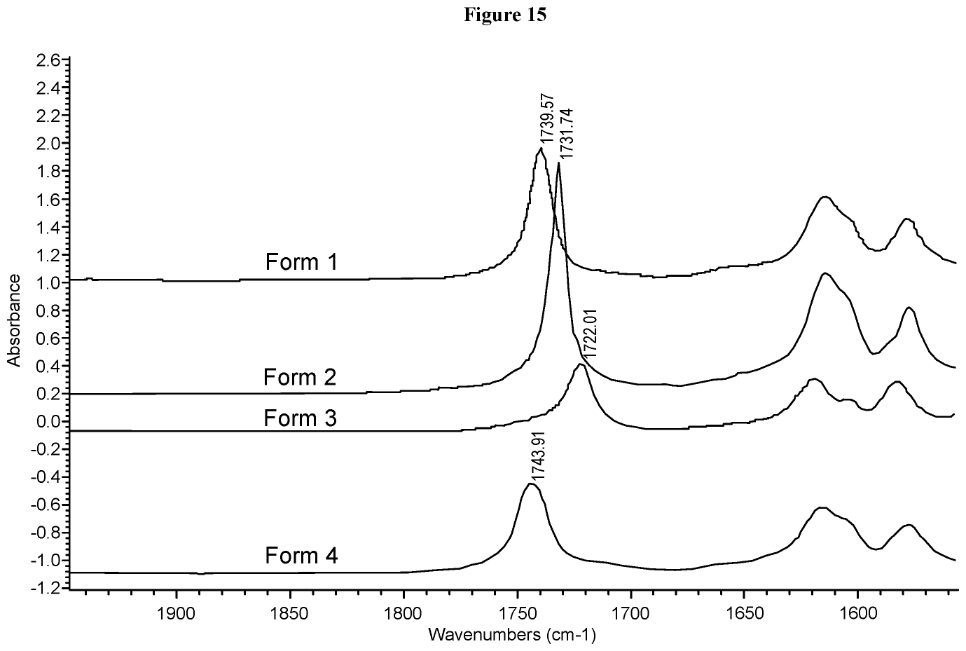

The partial Fourier Transform Infrared (FTIR) pattern overlay for crystalline Forms 1, 2, 3, and 4 of Compound I is displayed in FIG. 15. The FTIR spectrum for Crystalline Form 1 has a peak at about 1739.6 cm⁻¹. The FTIR spectrum for Crystalline Form 2 has a peak at about 1731.7 cm⁻¹. The FTIR spectrum for Crystalline Form 3 has a peak at about 1722.0 cm⁻¹. The FTIR spectrum for Crystalline Form 4 has a peak at about 1743.9 cm⁻¹.

### Example 13: Fourier Transform Raman Spectroscopy

Raman spectra were acquired on a Raman module interfaced to a Nicolet 6700 IR spectrophotometer (Thermo Nicolet) equipped with an indium gallium arsenide (InGaAs) detector. Wavelength verification was performed using sulfur and cyclohexane. Each sample was prepared for analysis by placing the sample into a 13 mm diameter stainless steel cup and leveling the material. A Thermo Nicolet Step-and-Repeat accessory was used to spin the cup during data acquisition. Three spectra were collected for each sample from outer to inner rings of the sample cup. Approximately 0.5 W of Nd:YVO4 laser power (1064 nm excitation wavelength) was used to irradiate the sample. Each spectrum consists of 512 co-added scans with a spectral resolution of 2 cm⁻¹. The three spectra for each sample were averaged using Omnic v7.2 (ThermoElectron).

Raman peak position variabilities are given to within ±2 cm⁻¹, based on the observed sharpness of the peaks picked and acquisition of data using a 1 cm⁻¹ data point spacing (2 cm⁻¹ resolution). The peak picking was performed using OMNIC software, version 7.2, Thermo Electron Corporation. Observed Peaks include all Raman peaks for a given form, with the exclusion of very weak intensity peaks and broad peaks with poorly defined maxima.

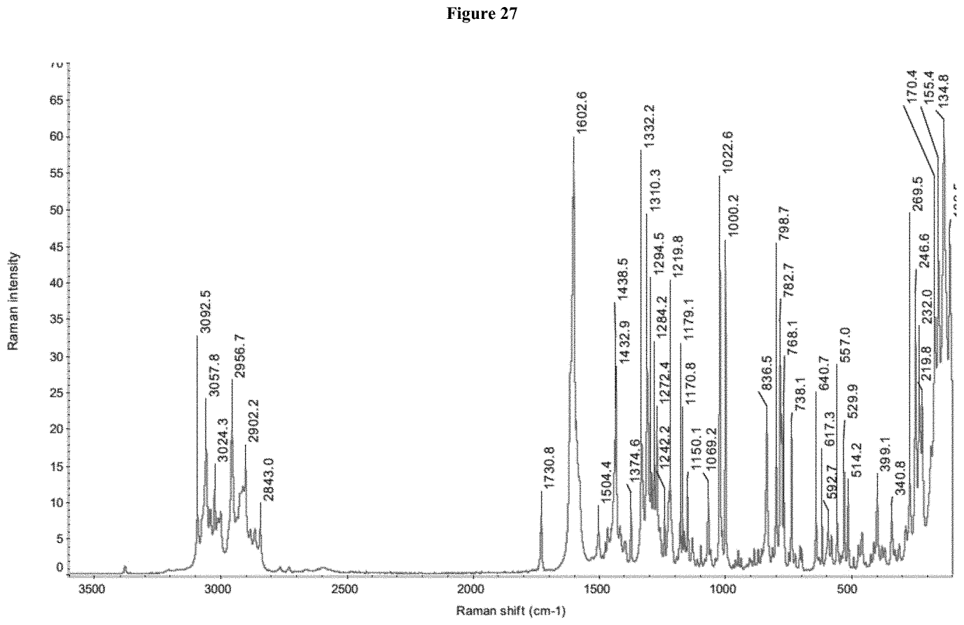

The Raman spectrum for Form 1 is displayed in FIG. 27.

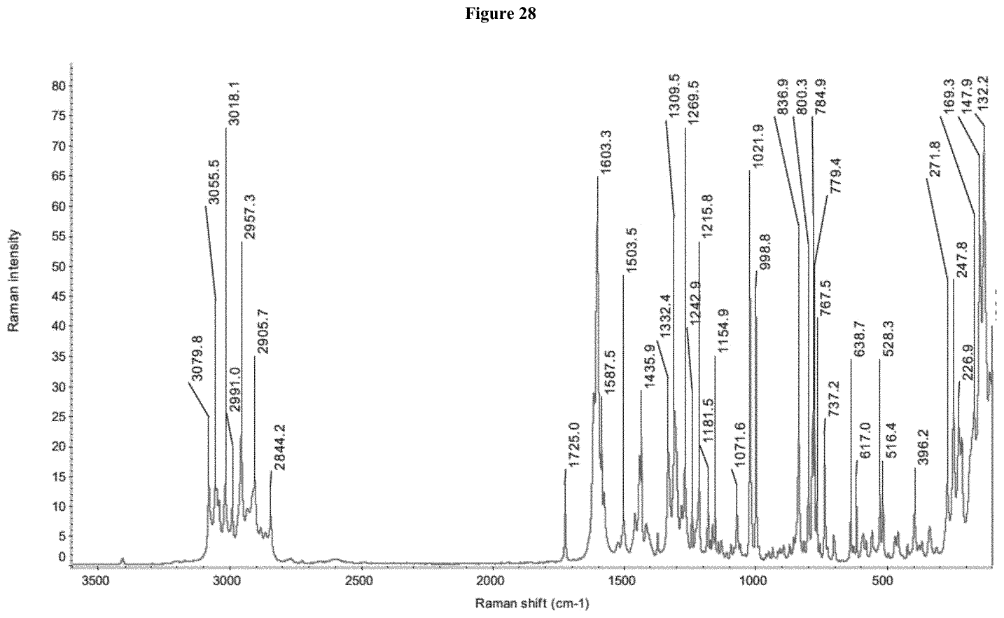

The Raman spectrum for Form 2 is displayed in FIG. 28.

### Example 14: Solid State Nuclear Magnetic Resonance (ssNMR) Spectroscopy

All spectra were acquired using a Bruker DRX500 spectrometer, equipped with a 11.7 Tesla magnet and a 4 mm diameter solid-state probe. The following parameters were employed:

| Observation nucleus | ¹³C | |
|---|---|---|
| Observation frequency | 125.77 | MHz |
| Complex data points | 2716 zero-filled to 4096 | |
| Spectral width | 34.0 | kHz |
| Acquisition time | 40 | ms |
| Number of dummy transients | 2 | |
| Number of transients | 2048 | |
| Relaxation delay | 11.0 s for Form 1 | |
| | 20.0 s for Form 2 | |
| | 12.0 s for Form 3 | |
| | 11.0 s for Amorphous | |
| Contact time | 5.0 ms for Form 1 | |
| | 3.0 ms for Form 2 | |
| | 2.0 ms for Form 3 | |
| | 3.0 ms for Amorphous | |
| π/2 proton pulse length | 2.9 | μs |
| ¹H decoupling | TPPM-15 | |
| Sample rotation rate | 14.0 | kHz |

| | |
|---|---|
| Temperature | Ambient |

All spectra are referenced indirectly with respect to tetramethylsilane using the high frequency signal of adamantane. All samples were packed into 4 mm OD rotors constructed of zirconia, fitted with a Kel-F drive cap. A Gaussian convolution was applied to the free induction decay prior to Fourier transformation; GB=0.035 and LB=−10.0 Hz.

### Characterization of Crystalline Form 1 of Compound I

The ssNMR spectrum for crystalline Form 1 of Compound I is displayed in FIG. 4. Resonances that are characteristic of Form 1 are listed below:

δc/ppm: 23.35, 36.40, 44.12, 45.70, 54.41, 65.40, 71.58, 110.97, 114.45, 121.00, 124.43, 126.78, 127.42, 131.27, 136.47, 138.94, 142.61, 148.68, 152.19, 172.07, 174.59

### Characterization of Crystalline Form 2 of Compound I

The ssNMR spectrum for crystalline Form 2 of Compound I is displayed in FIG. 8. Resonances that are characteristic of Form 2 are listed below:

δc/ppm: 20.59, 37.04, 44.03, 46.84, 55.25, 66.34, 71.74, 111.25, 116.90, 122.48, 123.63, 126.39, 128.34, 131.33, 136.78, 137.69, 141.73, 149.44, 153.68, 172.82, 175.49

### Characterization of Crystalline Form 3 of Compound I

The ssNMR spectrum for crystalline Form 3 of Compound I is displayed in FIG. 12. Resonances that are characteristic of Form 3 are listed below:

♦c/ppm: 21.72^{#}, 22.23^{#}, 43.81, 46.00, 54.01, 64.56, 67.67, 109.22, 110.33, 119.58, 122.99, 126.71, 130.28^{#}, 138.46^{#}, 139.68, 140.34, 143.63, 144.25, 146.87, 150.90, 168.32, 176.47

# broadened or split signals whose shape or chemical shift may vary.

### Characterization of Amorphous Form of Compound I

The ssNMR spectrum for amorphous form of Compound I is displayed in FIG. 16.

### Example 15: Stability of Solid State Forms

The physical stability of Forms 1, 2, and 3 was investigated at 80° C./75% RH in order to determine if interconversion was observed. The samples were examined by FTIR after stressing 1 week in open glass vials.

No changes in the FTIR spectrums were observed for any of the forms, suggesting that these forms are relatively stable in the solid state.

### Example 16: Solubility Studies

The solubility of the different polymorphs was determined at pH 7.4 in phosphate buffer at 25° C. Samples were analyzed as a function of time for each form to determine the equilibrium values. The residual solids from each sample were analyzed to verify that the form was unchanged during the experiment. The concentration (mg/mL) versus time data is listed below for each form:

| | 1 hr | 2 hr | 3 hr | 24 hr |
|---|---|---|---|---|
| Form 1 | 0.042 | 0.041 | 0.042 | 0.042 |
| Form 2 | 0.034 | 0.039 | 0.043 | 0.057* |
| Form 3 | 0.083 | 0.093 | 0.095 | 0.097 |
| Form 4 | 0.079 | 0.089 | 0.105 | 0.102 |

*additional time point confirmed equilibrium

The equilibrium solubility values at 24 hours show Forms 3 and 4 to be more than double the solubility for Form 1. The 24 hour result for Form 2 was more than 30% greater than Form 1.

It should be noted that analysis of the residual solids showed no polymorphic conversion during the course of the experiments. The data for Forms 3 and 4 are equivalent within experimental error.

### Example 17: Single Crystal X-Ray Diffraction (SCXRD) of Crystalline Form 1 of Compound I

Crystallization of Compound I from propyl acetate yielded a crystal—0.5*0.04*0.02 mm³ in size—which was sealed in a Lindemann-glass capillary. X-ray diffraction data were collected on a Bruker/AXS three circle diffractometer, equipped with a SMART APEX area-detector, a low temperature device (model LT 2) and a molybdenum-K_{α} rotating anode generator, operated at 50 kV/120 mA and adjusted to a fine-focus of 0.5×5 mm². Data frames were collected using the program package SMART V 5.628 (Bruker AXS, 2001), applying ω-scans with step widths of 0.3° and an exposure time of 60 seconds. Data processing with the program SAINT+Release 6.45 (Bruker AXS, 2003) yielded 6452 reflections (ϑₘᵢₙ=2.04, ϑₘₐₓ=28.06; −8<h<8, −7<k<13, −22<1<22) of which 4753 reflections were unique (Rᵢₙₜ=0.0829, R_{σ}=0.2353). Refinement of the cell parameters was performed using 720 reflections. The phase problem was solved with direct methods by the XS module of SHELXTL 6.14 (Bruker AXS, 2000).

The structure was refined by least-squares methods (minimization of (Fₒ²−F_{c}²)²) using the XL module of SHELXTL 6.14 (Bruker AXS, 2000). The positions of all H atoms were experimentally determined from a difference Fourier synthesis map, S_{goodness of fit}=0.780, R_{all data}=0.2189 (R_{obs. data}=0.0536 for 1479 reflections with |F_{obs}|>4σ, wR2_{all data}=0.1080, wR2_{obs. data}=0.0759). The largest unassigned peaks in the difference map correspond to −0.193 versus +0.162 electrons per Å³. The average estimated standard deviation (e.s.d.) of a C—C bond is 0.005 Å, that of an O—C bond 0.004 Å, that of an N—C bond 0.004 Å and that of a C—H bond 0.03 Å. The average e.s.d. of C—C—C bond angles is 0.4 and that of C—C—C—C torsion angles 0.5°.

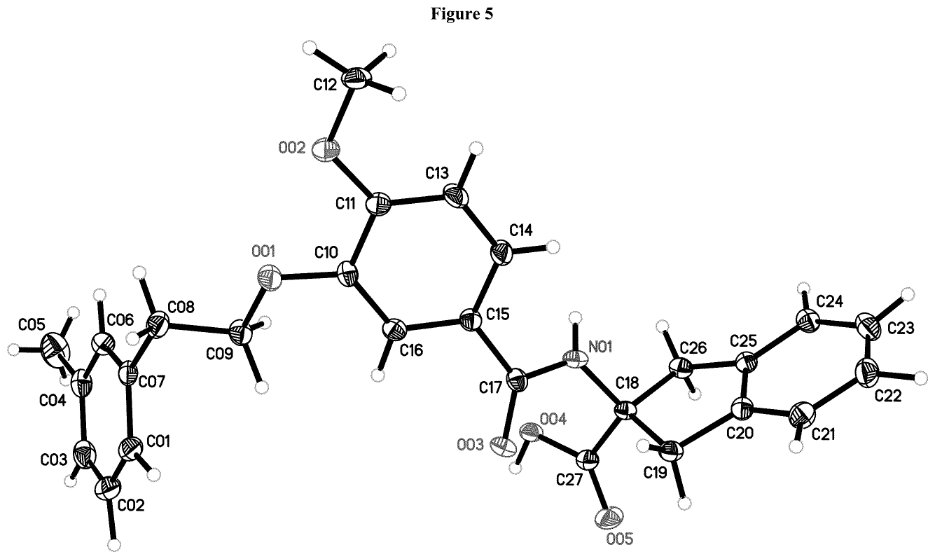

The crystal structure of Crystalline Form 1 of Compound I was determined at 293 K and a summary of the structural data can be found in Table 1 and Table 2. The molecular structure is shown in FIG. 5.

TABLE 1
Crystal Data of Compound I (Form 1) at 293 K

| | |
|---|---|
| Crystal System | triclinic |
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |
| Model Quality | R_{obs. data} = 5.36% |

TABLE 2
Atomic coordinates and equivalent isotropic displacement parameters
[Å] for Compound I (Form 1) at 293 K

| | x | Y | z | U(eq)* |
|---|---|---|---|---|
| O01 | 0.5030(4) | 0.4696(2) | −0.24147(13) | 0.0519(8) |
| O02 | 0.1244(4) | 0.3734(2) | −0.21202(13) | 0.0517(7) |
| O03 | 0.8430(4) | 0.7934(2) | 0.03104(13) | 0.0437(7) |
| O04 | 0.8283(4) | 1.0809(2) | 0.02552(15) | 0.0441(7) |
| O05 | 1.0255(4) | 1.1365(3) | 0.14238(14) | 0.0665(9) |
| N01 | 0.5633(5) | 0.8827(3) | 0.06678(19) | 0.0418(9) |
| C01 | 1.0831(7) | 0.5910(4) | −0.3552(2) | 0.0543(12) |
| C02 | 1.2317(7) | 0.6751(5) | −0.3842(3) | 0.0661(14) |
| C03 | 1.1721(8) | 0.7334(5) | −0.4423(3) | 0.0654(15) |
| C04 | 0.9651(7) | 0.7091(4) | −0.4737(2) | 0.0557(12) |
| C05 | 0.9012(14) | 0.7688(9) | −0.5392(4) | 0.092(2) |
| C06 | 0.8190(7) | 0.6242(4) | −0.4438(2) | 0.0516(12) |
| C07 | 0.8725(6) | 0.5657(4) | −0.3858(2) | 0.0435(10) |
| C08 | 0.7044(8) | 0.4798(5) | −0.3518(2) | 0.0552(13) |
| C09 | 0.6788(7) | 0.5492(4) | −0.2681(2) | 0.0478(12) |
| C10 | 0.4477(6) | 0.5258(4) | −0.1675(2) | 0.0403(10) |
| C11 | 0.2397(6) | 0.4736(3) | −0.1521(2) | 0.0438(10) |
| C12 | −0.0778(7) | 0.3071(5) | −0.1940(3) | 0.0548(12) |
| C13 | 0.1733(7) | 0.5270(4) | −0.0805(2) | 0.0531(12) |
| C14 | 0.3017(6) | 0.6301(4) | −0.0243(2) | 0.0515(12) |
| C15 | 0.5061(5) | 0.6793(3) | −0.0386(2) | 0.0379(10) |
| C16 | 0.5770(6) | 0.6257(4) | −0.1102(2) | 0.0400(10) |
| C17 | 0.6506(6) | 0.7874(4) | 0.0212(2) | 0.0388(10) |
| C18 | 0.6849(5) | 0.9914(3) | 0.1310(2) | 0.0361(9) |
| C19 | 0.7593(6) | 0.9353(5) | 0.1986(2) | 0.0432(11) |
| C20 | 0.5965(5) | 0.9512(3) | 0.2570(2) | 0.0402(10) |
| C21 | 0.5709(7) | 0.8992(5) | 0.3221(3) | 0.0529(12) |
| C22 | 0.4184(7) | 0.9324(5) | 0.3719(3) | 0.0613(13) |
| C23 | 0.2946(8) | 1.0159(5) | 0.3560(3) | 0.0607(14) |
| C24 | 0.3156(6) | 1.0695(4) | 0.2899(2) | 0.0468(11) |
| C25 | 0.4722(6) | 1.0356(3) | 0.2408(2) | 0.0378(10) |
| C26 | 0.5351(6) | 1.0837(4) | 0.1691(2) | 0.0405(10) |
| C27 | 0.8654(6) | 1.0758(4) | 0.1007(2) | 0.0429(10) |
| H1 | 0.448(4) | 0.886(3) | 0.0505(17) | 0.025(11) |
| H4 | 0.953(7) | 1.131(4) | 0.016(2) | 0.112(18) |
| H01 | 1.122(4) | 0.543(3) | −0.3064(17) | 0.050(10) |
| H02 | 1.390(6) | 0.700(3) | −0.357(2) | 0.089(14) |
| H03 | 1.276(5) | 0.791(3) | −0.4616(18) | 0.061(12) |
| H051 | 0.924(8) | 0.726(5) | −0.582(3) | 0.12(3) |
| H052 | 0.999(10) | 0.857(6) | −0.534(4) | 0.24(4) |
| H053 | 0.776(7) | 0.777(5) | −0.536(3) | 0.13(3) |
| H06 | 0.680(5) | 0.607(3) | −0.4665(17) | 0.041(11) |
| H081 | 0.745(5) | 0.392(3) | −0.3537(19) | 0.062(14) |
| H082 | 0.571(5) | 0.446(3) | −0.3879(18) | 0.064(12) |
| H091 | 0.647(5) | 0.645(3) | −0.2638(17) | 0.052(12) |
| H092 | 0.815(5) | 0.570(3) | −0.2255(19) | 0.073(12) |
| H121 | −0.175(6) | 0.376(4) | −0.183(2) | 0.086(15) |
| H122 | −0.130(5) | 0.245(3) | −0.244(2) | 0.079(14) |
| H123 | −0.054(6) | 0.267(4) | −0.136(3) | 0.131(18) |
| H13 | 0.035(5) | 0.500(3) | −0.073(2) | 0.076(14) |
| H14 | 0.256(4) | 0.664(3) | 0.0241(17) | 0.044(11) |
| H16 | 0.716(4) | 0.667(2) | −0.1171(14) | 0.025(9) |
| H191 | 0.750(4) | 0.844(3) | 0.1739(17) | 0.041(11) |
| H192 | 0.902(5) | 0.999(3) | 0.2276(15) | 0.043(9) |
| H21 | 0.651(5) | 0.842(3) | 0.3281(19) | 0.047(13) |
| H22 | 0.399(5) | 0.900(3) | 0.426(2) | 0.085(13) |
| H23 | 0.181(6) | 1.031(4) | 0.385(2) | 0.082(15) |
| H24 | 0.227(5) | 1.130(3) | 0.2714(17) | 0.047(11) |
| H261 | 0.611(5) | 1.188(3) | 0.1875(16) | 0.051(10) |
| H262 | 0.427(5) | 1.084(3) | 0.1328(17) | 0.051(12) |

*U(eq) is defined as one third of the trace of the orthogonalized Uij tensor.

### Example 18: Single Crystal X-Ray Diffraction (SCXRD) of Crystalline Form 2 of Compound I

Crystallization of Compound I from N-methyl-2-pyrrolidone/methanol yielded a crystal—0.6*0.2*0.2 mm³ in size—which was sealed in a Lindemann-glass capillary. X-ray diffraction data were collected on a Bruker/AXS three circle diffractometer, equipped with a SMART APEX area-detector, a low temperature device (model LT 2) and a copper-K_{α} microfocus generator, operated at 45 kV/650 μA and a focusing beam Montel multilayer optic with an image focus spot diameter of ˜250 μm (Wiesmann et al., 2007). Data frames were collected using the program package SMART V 5.628 (Bruker AXS, 2001), applying ω-scans with step widths of 0.3° and an exposure time of 5 seconds. Data processing with the program SAINT+Release 6.45 (Bruker AXS, 2003) yielded 23571 reflections (ϑₘᵢₙ=2.80, ϑₘₐₓ=69.16; −7<h<6, −28<k<26, −34<l<38) of which 4163 reflections were unique (Rᵢₙₜ=0.0242, R_{σ}=0.0190). Refinement of the cell parameters was performed using the 99 local cell parameter determinations observed during data integration. An empirical absorption correction has been applied using the program SADABS, a module of SAINT 6.45 (Bruker AXS, 2003). The phase problem was solved with direct methods by the XS module of SHELXTL 6.14 (Bruker AXS, 2000).

The structure was refined by least-squares methods (minimization of (Fₒ²−F_{c}²)²) using the XL module of SHELXTL 6.14 (Bruker AXS, 2000). The positions of all H atoms were experimentally determined from a difference Fourier synthesis map, S_{goodness of fit}=1.039, R_{all data}=0.0490 (R_{obs. data}=0.0379 for 3283 reflections with |F_{obs}|>4σ, wR2_{all data}=0.1041, wR2_{obs. data}=0.0971). The largest unassigned peaks in the difference map correspond to −0.179 versus +0.185 electrons per Å³. The average estimated standard deviation (e.s.d.) of a C—C bond is 0.002 Å, that of an O—C bond 0.002 Å, that of an N—C bond 0.002 Å and that of a C—H bond 0.02 Å. The average e.s.d. of C—C—C bond angles is 0.2 and that of C—C—C—C torsion angles 0.2°.

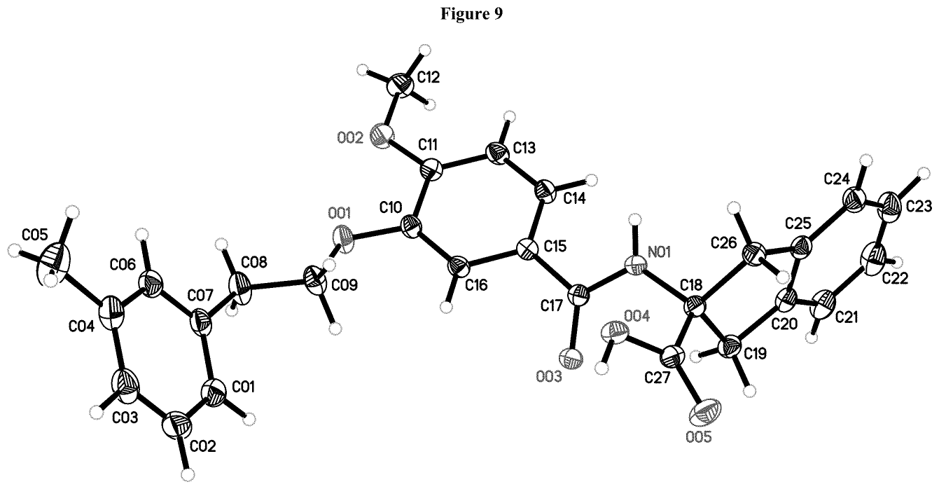

The crystal structure of Crystalline Form 2 of Compound I was determined at 293 K and a summary of the structural data can be found in Table 3 and Table 4. The molecular structure is shown in FIG. 9.

TABLE 3
Crystal Data of Compound I (Form 2) at 293 K

| | |
|---|---|
| Crystal System | orthorhombic |
| Space Group | Pbca; Z = 8 |
| a (Å) | 6.2823(10) |
| b (Å) | 23.285(4) |
| c (Å) | 31.614(6) |
| α (°) | 90.00° |
| β (°) | 90.00° |
| γ (°) | 90.00° |
| V (Å³) | 4624.5(14) |
| Calculated Density (Mg/m³) | 1.280 |
| Unique Reflections | 4163 |
| Model Quality | R_{obs. data} = 3.79% |

TABLE 4
Atomic coordinates and equivalent isotropic displacement parameters
[Å] for Compound I (Form 2) at 293 K

| | x | Y | z | U(eq)* |
|---|---|---|---|---|
| O01 | 0.64153(19) | 0.50661(5) | 0.18516(3) | 0.0611(3) |
| O02 | 0.28437(18) | 0.45533(5) | 0.19955(3) | 0.0633(3) |
| O03 | 0.90062(15) | 0.41927(4) | 0.04333(3) | 0.0463(3) |
| O04 | 0.80055(16) | 0.50310(4) | −0.02774(3) | 0.0502(3) |
| O05 | 0.9886(2) | 0.44424(5) | −0.06867(4) | 0.0775(4) |
| N01 | 0.5874(2) | 0.41538(5) | 0.00862(4) | 0.0430(3) |
| C01 | 1.1636(3) | 0.63689(10) | 0.18246(6) | 0.0685(5) |
| C02 | 1.2638(4) | 0.68761(11) | 0.17096(6) | 0.0805(6) |
| C03 | 1.1664(4) | 0.73952(10) | 0.17789(6) | 0.0800(6) |
| C04 | 0.9689(3) | 0.74237(8) | 0.19659(6) | 0.0720(5) |
| C05 | 0.8583(8) | 0.79928(13) | 0.20384(14) | 0.1180(11) |
| C06 | 0.8710(3) | 0.69112(8) | 0.20812(5) | 0.0636(5) |
| C07 | 0.9644(3) | 0.63853(7) | 0.20097(5) | 0.0578(4) |
| C08 | 0.8452(4) | 0.58411(10) | 0.21203(6) | 0.0732(6) |
| C09 | 0.7850(3) | 0.55127(9) | 0.17367(5) | 0.0629(5) |
| C10 | 0.5684(2) | 0.47210(6) | 0.15318(4) | 0.0460(3) |
| C11 | 0.3731(2) | 0.44413(6) | 0.16110(4) | 0.0472(4) |
| C12 | 0.1001(3) | 0.42334(10) | 0.21130(7) | 0.0692(5) |
| C13 | 0.2878(3) | 0.40883(7) | 0.13037(5) | 0.0517(4) |
| C14 | 0.3910(2) | 0.40118(7) | 0.09210(5) | 0.0493(4) |
| C15 | 0.5845(2) | 0.42789(6) | 0.08447(4) | 0.0416(3) |
| C16 | 0.6729(2) | 0.46302(6) | 0.11559(4) | 0.0444(3) |
| C17 | 0.7039(2) | 0.42086(5) | 0.04418(4) | 0.0404(3) |
| C18 | 0.6857(2) | 0.40508(6) | −0.03269(4) | 0.0422(3) |
| C19 | 0.7875(3) | 0.34474(7) | −0.03372(5) | 0.0502(4) |
| C20 | 0.6175(2) | 0.30717(6) | −0.05197(4) | 0.0481(4) |
| C21 | 0.6098(4) | 0.24762(8) | −0.05344(6) | 0.0662(5) |
| C22 | 0.4395(4) | 0.22158(9) | −0.07369(6) | 0.0783(6) |
| C23 | 0.2804(4) | 0.25389(9) | −0.09190(6) | 0.0736(6) |
| C24 | 0.2872(3) | 0.31333(8) | −0.09077(5) | 0.0579(4) |
| C25 | 0.4573(2) | 0.33957(6) | −0.07050(4) | 0.0459(3) |
| C26 | 0.5073(3) | 0.40272(7) | −0.06663(5) | 0.0471(4) |
| C27 | 0.8436(2) | 0.45215(6) | −0.04449(5) | 0.0479(4) |
| H1 | 0.453(3) | 0.4252(7) | 0.0094(5) | 0.054(5) |
| H4 | 0.906(3) | 0.5274(9) | −0.0346(6) | 0.087(6) |
| H01 | 1.231(3) | 0.5999(10) | 0.1763(6) | 0.084(6) |
| H02 | 1.396(4) | 0.6848(9) | 0.1581(7) | 0.097(7) |
| H03 | 1.243(4) | 0.7768(10) | 0.1684(6) | 0.100(7) |
| H051 | 0.899(7) | 0.8235(19) | 0.1837(13) | 0.20(2) |
| H052 | 0.835(7) | 0.8063(18) | 0.2320(14) | 0.208(19) |
| H053 | 0.704(11) | 0.798(2) | 0.1966(18) | 0.28(3) |
| H06 | 0.731(3) | 0.6930(8) | 0.2207(6) | 0.076(6) |
| H081 | 0.719(4) | 0.5932(11) | 0.2274(8) | 0.121(9) |
| H082 | 0.919(3) | 0.5607(10) | 0.2329(7) | 0.096(7) |
| H091 | 0.733(3) | 0.5726(9) | 0.1502(7) | 0.086(6) |
| H092 | 0.923(4) | 0.5349(9) | 0.1618(7) | 0.103(7) |
| H121 | −0.019(4) | 0.4312(9) | 0.1902(7) | 0.092(7) |
| H122 | 0.063(3) | 0.4362(9) | 0.2379(7) | 0.092(7) |
| H123 | 0.136(3) | 0.3791(10) | 0.2109(6) | 0.090(6) |
| H13 | 0.158(3) | 0.3900(7) | 0.1360(5) | 0.059(5) |
| H14 | 0.332(3) | 0.3756(7) | 0.0716(5) | 0.054(4) |
| H16 | 0.803(2) | 0.4808(6) | 0.1100(4) | 0.046(4) |
| H191 | 0.835(3) | 0.3324(7) | −0.0053(5) | 0.060(5) |
| H192 | 0.912(3) | 0.3445(7) | −0.0524(5) | 0.065(5) |
| H21 | 0.717(3) | 0.2274(8) | −0.0407(6) | 0.072(6) |
| H22 | 0.433(3) | 0.1802(9) | −0.0748(6) | 0.083(6) |
| H23 | 0.157(3) | 0.2352(9) | −0.1048(6) | 0.090(6) |
| H24 | 0.174(3) | 0.3357(8) | −0.1036(6) | 0.070(5) |
| H261 | 0.566(2) | 0.4176(6) | −0.0943(5) | 0.058(4) |
| H262 | 0.385(3) | 0.4262(7) | −0.0586(5) | 0.053(4) |

*U(eq) is defined as one third of the trace of the orthogonalized Uij tensor

The experimentally determined powder diffraction pattern agrees with the one calculated from the crystal structure.

### Example A-1: Parenteral Pharmaceutical Composition

To prepare a parenteral pharmaceutical composition suitable for administration by injection (subcutaneous, intravenous), 1-100 mg of Compound I, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2: Oral Solution

To prepare a pharmaceutical composition for oral delivery, a sufficient amount of Compound I, or a pharmaceutically acceptable salt thereof, is added to water (with optional solubilizer(s),optional buffer(s) and taste masking excipients) to provide a 20 mg/mL solution.

### Example A-3: Oral Tablet

A tablet is prepared by mixing 20-50% by weight of Compound I, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of Compound I, or a pharmaceutically acceptable salt thereof, is optionally mixed with starch or other suitable powder blends. The mixture is incorporated into an oral dosage unit such as a hard gelatin capsule, which is suitable for oral administration.

In another embodiment, 10-500 mg of a compound disclosed herein, or a pharmaceutically acceptable salt thereof, is placed into Size 4 capsule, or size 1 capsule (hypromellose or hard gelatin) and the capsule is closed.

The examples and embodiments described herein are for illustrative purposes only and various modifications or changes suggested to persons skilled in the art are to be included within the spirit and purview of this application and scope of the appended claims.

We claim:

1. Crystalline Form 1 of 2-(4-methoxy-3-(3-methylphenethoxy)benzamido)-2,3-dihydro-1H-indene-2-carboxylic acid (Compound I), characterized as having an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation.

2. Crystalline Form 1 of 2-(4-methoxy-3-(3-methylphenethoxy)benzamido)-2,3-dihydro-1H-indene-2-carboxylic acid (Compound I), characterized as having an X-ray powder diffraction (XRPD) pattern substantially the same as shown in FIG. 1, as measured using Cu (Kα) radiation.

3. The crystalline form of claim 1, wherein the crystalline Form 1 of Compound I is further characterized as having a Fourier Transform IR Spectroscopy (FTIR) pattern with a peak at about 1739.6 cm⁻¹.

4. The crystalline form of claim 1, wherein the crystalline Form 1 of Compound I is further characterized as having a Solid State ¹³Carbon Nuclear Magnetic Resonance (ssNMR) spectrum substantially the same as shown in FIG. 4.

5. The crystalline form of claim 1, wherein the crystalline Form 1 of Compound I is further characterized as having a Solid State ¹³Carbon Nuclear Magnetic Resonance (ssNMR) spectrum characterized by resonances (δc) at 23.35, 124.43, 126.78, 127.42, and 136.47 ppm.

6. The crystalline form of claim 1, wherein the crystalline Form 1 of Compound I is further characterized as having a Differential Scanning calorimetry (DSC) thermogram substantially the same as shown in FIG. 2.

7. The crystalline form of claim 1, wherein the crystalline Form 1 of Compound I is further characterized as having a Differential Scanning calorimetry (DSC) thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

8. The crystalline form of claim 2, wherein the crystalline Form 1 of Compound I is further characterized as having a Fourier Transform IR Spectroscopy (FTIR) pattern with a peak at about 1739.6 cm⁻¹.

9. The crystalline form of claim 2, wherein the crystalline Form 1 of Compound I is further characterized as having a Solid State ¹³Carbon Nuclear Magnetic Resonance (ssNMR) spectrum substantially the same as shown in FIG. 4.

10. The crystalline form of claim 2, wherein the crystalline Form 1 of Compound I is further characterized as having a Solid State ¹³Carbon Nuclear Magnetic Resonance (ssNMR) spectrum characterized by resonances (δc) at 23.35, 124.43, 126.78, 127.42, and 136.47 ppm.

11. The crystalline form of claim 2, wherein the crystalline Form 1 of Compound I is further characterized as having a Differential Scanning calorimetry (DSC) thermogram substantially the same as shown in FIG. 2.

12. The crystalline form of claim 2, wherein the crystalline Form 1 of Compound I is further characterized as having a Differential Scanning calorimetry (DSC) thermogram with three endothermic events having: an onset at about 198.5° C. and a peak at about 200.4° C.; an onset at about 204.8° C. and a peak at about 205.8° C.; and an onset at about 213.9° C. and a peak at about 216.3° C.

13. The crystalline form of claim 1, wherein the crystalline Form 1 of Compound I has unit cell parameters substantially equal to the following at 293 K:

| Crystal System | triclinic |
|---|---|
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |

14. The crystalline form of claim 2, wherein the crystalline Form 1 of Compound I has unit cell parameters substantially equal to the following at 293 K:

| Crystal System | triclinic |
|---|---|
| Space Group | P-1; Z = 2 |
| a (Å) | 6.521(6) |
| b (Å) | 10.548(9) |
| c (Å) | 17.453(15) |
| α (°) | 104.080(16) |
| β (°) | 92.430(16) |
| γ (°) | 101.081(17) |
| V (Å³) | 1137.6(17) |
| Calculated Density (Mg/m³) | 1.301 |
| Unique Reflections | 4753 |

15. The crystalline form of claim 1, wherein the crystalline Form 1 of Compound I is anhydrous.

16. The crystalline form of claim 2, wherein the crystalline Form 1 of Compound I is anhydrous.

17. The crystalline form of claim 1, wherein the crystalline Form 1 of Compound I is substantially free of crystalline Form 2 of Compound I.

18. The crystalline form of claim 1, wherein the crystalline Form 1 of Compound I comprises less than 1% w/w of crystalline Form 2 of Compound I.

19. The crystalline form of claim 2, wherein the crystalline Form 1 of Compound I is substantially free of crystalline Form 2 of Compound I.

20. The crystalline form of claim 3, wherein the crystalline Form 1 of Compound I comprises less than 1% w/w of crystalline Form 2 of Compound I.

21. Amorphous phase of 2-(4-methoxy-3-(3-methylphenethoxy)benzamido)-2,3-dihydro-1H-indene-2-carboxylic acid (Compound I) characterized as having: an X-ray powder diffraction (XRPD) pattern showing a lack of crystallinity, and a Solid State ¹³Carbon Nuclear Magnetic Resonance (ssNMR) spectrum substantially the same as shown in FIG. 16.

22. A pharmaceutical composition comprising crystalline Form 1 of 2-(4-methoxy-3-(3-methylphenethoxy)benzamido)-2,3-dihydro-1H-indene-2-carboxylic acid (Compound I), and at least one pharmaceutically acceptable excipient;
wherein crystalline Form 1 of Compound I is characterized as having an X-ray powder diffraction (XRPD) pattern with peaks at 5.2±0.2° 2-Theta, 9.0±0.2° 2-Theta, 14.4±0.2° 2-Theta, and 17.7±0.2° 2-Theta, as measured using Cu (Kα) radiation; and
wherein the pharmaceutical composition is in the form of a solid form pharmaceutical composition.

23. The pharmaceutical composition of claim 22, wherein the pharmaceutical composition is in the form of a tablet, a pill, or a capsule.

24. The pharmaceutical composition of claim 22, wherein the pharmaceutical composition is in the form of a tablet and comprises about 50 mg to about 300 mg of crystalline Form 1 of Compound I.

25. The pharmaceutical composition of claim 22, wherein the pharmaceutical composition is in the form of a tablet and comprises about 150 mg of crystalline Form 1 of Compound I.

26. The pharmaceutical composition of claim 22, wherein the crystalline Form 1 of Compound I is substantially free of crystalline Form 2 of Compound I.

27. The pharmaceutical composition of claim 22, wherein the crystalline Form 1 of Compound I comprises less than 1% w/w of crystalline Form 2 of Compound I.
